SEQUENCE LISTING (7 sequences)

SEQ ID NO: 1 - protein, 210 aa, Homo sapiens
  SITE 1..210: note = full-length human FXN protein
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
  SITE 1..130: note = mature human FXN protein
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Homo sapiens
  SITE 1..80: note = mitochondrial targeting sequence of human FXN protein
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, Human immunodeficiency virus
  SITE 1..11: note = HIV-TAT peptide
YGRKKRRQRRR

SEQ ID NO: 5 - protein, 224 aa, synthetic construct
  REGION 1..224: note = Synthetic: TAT-FXN fusion protein
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 6 - protein, 17 aa, Homo sapiens
  SITE 1..17: note = peptide derived from mature human FXN protein
SGTLGHPGSLDETTYER

SEQ ID NO: 7 - protein, 12 aa, Homo sapiens
  SITE 1..12: note = peptide derived from mature human FXN protein
LGGDLGTYVINK

(19) United States
(12) Patent Application Publication
(10) Pub. No.: US 2025/0276037 A1
(43) Pub. Date: Sep. 4, 2025

(54) METHODS OF TREATING FRIEDREICH'S ATAXIA

(71) Applicant: Larimar Therapeutics, Inc., Bala Cynwyd, PA (US)

(72) Inventors: Carole Sue Ben-Maimon, Merion Station, PA (US); Russell Clayton, Fernandina Beach, FL (US)

(21) Appl. No.: 19/051,988

(22) Filed: Feb. 12, 2025

Related U.S. Application Data
(60) Provisional application No. 63/734,702, filed on Dec. 16, 2024.
(60) Provisional application No. 63/718,993, filed on Nov. 11, 2024.
(60) Provisional application No. 63/655,286, filed on Jun. 3, 2024.
(60) Provisional application No. 63/552,633, filed on Feb. 12, 2024.

(51) Int. Cl. A61K 38/17; A61K 38/16; A61P 25/28

(52) U.S. Cl. CPC A61K 38/1709; A61K 38/162; A61P 25/28

(57) ABSTRACT

The present disclosure provides methods of increasing level of frataxin (FXN) in an FXN-decifient subject that comprise administering to the subject a TAT-FXN fusion protein once daily at a dose of about 25 mg or about 50 mg for at least 14 days. The present disclosure also provides methods of treating Friedreich's Ataxia (FRDA) in a subject that comprise administering to the subject a TAT-FXN fusion protein once daily at a dose of about 25 mg or about 50 mg for at least 14 days.

### RELATED APPLICATIONS

This application claims priority to U.S. Provisional Application No. 63/734,702, filed on Dec. 16, 2024; U.S. Provisional Application No. 63/718,993, filed on Nov. 11, 2024; U.S. Provisional Application No. 63/655,286, filed on Jun. 3, 2024; and U.S. Provisional Application No. 63/552,633, filed on Feb. 12, 2024. The entire contents of each of the foregoing applications are hereby incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 11, 2025, is named “130197-01705.xml” and is 10,305 bytes in size. The sequence listing contained in this .XML file is part of the specification and is hereby incorporated by reference herein in its entirety.

### INTRODUCTION

Mitochondrial diseases are a group of disorders caused by dysfunctional mitochondria, the cellular organelles that store potential energy in the form of adenosine triphosphate (ATP) molecules and are found in every cell of the human body except mature red blood cells.

Friedreich's Ataxia (FRDA) is the most common inherited ataxia in humans and results from a deficiency of the mitochondrial protein frataxin (FXN), and specifically human frataxin (hFXN). FRDA is a rare disease with an estimated incidence of 1:29,000, a carrier frequency of ˜1:85, and about 4,000-5,000 reported cases in the United States. FRDA is a progressive multisystem disease, typically beginning in mid-childhood. Subjects suffer from multiple symptoms, including progressive neurologic and cardiac dysfunction. Other clinical findings can include scoliosis, fatigue, diabetes, visual impairment, and hearing loss.

Inheritance is autosomal recessive and is predominantly caused by an inherited GAA triplet expansion in the first intron of both alleles of the hFXN gene. This triplet expansion causes transcriptional repression of the FRDA gene, which results in the production of very small amounts of hFXN in subjects. Subjects who are heterozygous for hFXN mutation typically have hFXN levels at ˜50% of normal but are phenotypically normal. Levels of hFXN of ˜45-70 pg/μl and ˜5-25 pg/μl in whole blood of heterozygotes and subjects afflicted with FRDA respectively have been shown to be stable over time. Most subjects with FRDA produce ˜20-40% of normal hFXN levels, depending on the tissue, sampling technique and assay considered (Deutsch et al., Molecular Genetics and Metabolism (2010), 101:238-245). Lower levels of hFXN are associated with earlier onset of disease, faster rate of disease progression and shorter time to loss of ambulation (Plasterer et al., PLoS ONE (2013), 8 (5): e63958; Rummey et al., EClinicalMedicine (2020), 18:100213). According to published data, the levels of hFXN in heterozygoes and subjects with FRDA is, respectively, 50.2% and 20.9% of hFXN levels of healthy controls in buccal cells; 75.3% and 32.2% in whole blood; 68.7% and 35.8% in peripheral blood mononuclear cells (PMBCs) and 64% and 29% in lymphoblastoid cells.

Currently, there is no FDA-approved treatment for FRDA. Antioxidants and iron chelation have not been overly effective, and, despite treatment, subjects typically experience progressive loss of motor control and die, cardiomyopathy being the primary cause of death.

Protein replacement therapy is a well-established approach to metabolic diseases, such as diabetes, lysosomal storage disorders and hemophilia. Work in subject-derived cellular and animal models has demonstrated that replacement of functional FXN can correct or improve the FRDA disease phenotype. However, there is a need in the art for reliable and efficient methods to measure clinical response and effectiveness of FXN replacement therapies.

### SUMMARY OF THE DISCLOSURE

In some aspects, the present disclosure provides a method of increasing level of frataxin (FXN) protein in an FXN deficient subject, the method comprising administering to the subject a TAT-FXN fusion protein at a dose of about 25 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased as compared to the level of FXN protein prior to the administration of the TAT-FXN fusion protein.

In some embodiments, the solid tissue sample is a skin biopsy. In some embodiments, the solid tissue sample is a buccal tissue.

In some embodiments, the TAT-FXN fusion protein is administered to the subject for at least 30 days, at least 60 days, or at least 90 days. In some embodiments, the TAT-FXN fusion protein is administered to the subject for at least 3 weeks, at least 4 weeks, at least 5 weeks, at least 6 weeks, at least 7 weeks, at least 8 weeks, at least 9 weeks, at least 10 weeks, or at least 12 weeks. In some embodiments, the TAT-FXN fusion protein is administered to the subject for more than 10 weeks.

In some embodiments, the level of FXN protein in a solid tissue sample obtained from the subject following administration of the TAT-FXN fusion protein is increased by at least about 10%, at least about 20%, at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%.

In some embodiments, the level of FXN protein in a solid tissue sample obtained from the subject following administration of the TAT-FXN fusion protein is increased to at least about 12.5%, at least about 25%, at least about 37.5%, at least about 50% or at least about 75% of the level of FXN protein in a healthy subject.

In some embodiments, the level of FXN protein in a buccal tissue sample following administration of the TAT-FXN fusion protein for at least 30 days is about 0.5 to about 3.0 pg/μg of total protein. In some embodiments, the level of FXN protein in a buccal tissue sample following administration of the TAT-FXN fusion protein for at least 60 days is about 1.0 to about 3.3 pg/μg of total protein. In some embodiments, the level of FXN protein in a buccal sample following administration of the TAT-FXN fusion protein for at least 90 days is about 1.4 to about 3.7 pg/μg of total protein.

In some embodiments, the level of FXN protein in a buccal tissue sample following administration of the TAT-FXN fusion protein for at least 30 days is increased by about 0.01 to about 1.5 pg/μg of total protein, as compared to the level of FXN protein prior to administration of the TAT-FXN fusion protein. In some embodiments, the level of FXN protein in a buccal tissue sample following administration of the TAT-FXN fusion protein for at least 60 days is increased by about 0.10 to about 1.8 pg/μg of total protein, as compared to the level of FXN protein prior to administration of the TAT-FXN fusion protein. In some embodiments, the level of FXN protein in a buccal tissue sample following administration of the TAT-FXN fusion protein for at least 90 days is increased by about 0.5 to about 2.6 pg/μg of total protein, as compared to the level of FXN protein prior to administration of the TAT-FXN fusion protein.

In some embodiments, the level of FXN protein in a buccal tissue sample following administration of the TAT-FXN fusion protein is increased to at least about 30% of the level of FXN protein in a healthy subject following administration of the TAT-FXN fusion protein for at least 30 days, at least 60 days, or at least 90 days.

In some embodiments, the level of FXN protein in a skin biopsy sample following administration of the TAT-FXN fusion protein for at least 30 days ranges from about 4.5 to about 8.5 pg/μg of total protein. In some embodiments, the level of FXN protein in a skin biopsy sample following administration of the TAT-FXN fusion protein for at least 90 days ranges from about 7.0 to about 10.5 pg/μg of total protein. In some embodiments, the level of FXN protein in a skin biopsy sample following administration of the TAT-FXN fusion protein for at least 30 days is increased by about 1.5 to about 6.0 pg/μg of total protein, as compared to the level of FXN protein prior to administration of the TAT-FXN fusion protein.

In some embodiments, the level of FXN protein in a skin biopsy sample following administration of the TAT-FXN fusion protein for at least 90 days is increased by about 4.2 to about 9.0 pg/μg of total protein, as compared to the level of FXN protein prior to administration of the TAT-FXN fusion protein. In some embodiments, the level of FXN protein in a skin biopsy sample following administration of the TAT-FXN fusion protein is increased to at least about 45% of the level of FXN protein in a healthy subject following administration of the TAT-FXN fusion protein for at least 30 days, or increased to at least about 70% of the level of FXN protein in a healthy subject following administration of the TAT-FXN fusion protein for at least 90 days.

In some aspects, the present disclosure provides a method of treating Friedreich's Ataxia in a subject in need thereof, the method comprising administering to the subject a TAT-FXN fusion protein at a dose of about 25 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased as compared to the level of FXN protein prior to the administration of the TAT-FXN fusion protein.

In some embodiments, the solid tissue sample is a skin biopsy. In some embodiments, the solid tissue sample is a buccal tissue.

In some embodiments, the TAT-FXN fusion protein is administered to the subject for at least 30 days, at least 60 days, or at least 90 days. In some embodiments, the TAT-FXN fusion protein is administered to the subject for at least 3 weeks, at least 4 weeks, at least 5 weeks, at least 6 weeks, at least 7 weeks, at least 8 weeks, at least 9 weeks, at least 10 weeks, or at least 12 weeks. In some embodiments, the TAT-FXN fusion protein is administered to the subject for more than 10 weeks.

In some embodiments, the level of TAT-FXN protein in a solid tissue sample obtained from the subject following administration of the TAT-FXN fusion protein is increased by at least about 10%, at least about 20%, at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%.

In some embodiments, the level of FXN protein in a solid tissue sample obtained from the subject following administration of the TAT-FXN fusion protein is increased to at least about 12.5%, at least about 25%, at least about 37.5%, at least about 50%, or at least about 70% of the level of FXN protein in a healthy subject.

In some embodiments the level of FXN protein in a buccal tissue sample following administration of the TAT-FXN fusion protein for at least 30 days is about 0.5 to about 3.0 pg/μg of total protein. In some embodiments, the level of FXN protein in a buccal tissue sample following administration of the TAT-FXN fusion protein for at least 60 days is about 1.0 to about 3.3 pg/μg of total protein. In some embodiments, the level of FXN protein in a buccal sample following administration of the TAT-FXN fusion protein for at least 90 days is about 1.4 to about 3.7 pg/μg of total protein.

In some embodiments, the level of FXN protein in a buccal tissue sample following administration of the TAT-FXN fusion protein for at least 30 days is increased by about 0.01 to about 1.5 pg/μg of total protein, as compared to the level of FXN protein prior to administration of the TAT-FXN fusion protein. In some embodiments, the level of FXN protein in a buccal tissue sample following administration of the TAT-FXN fusion protein for at least 60 days is increased by about 0.10 to about 1.8 pg/μg of total protein, as compared to the level of FXN protein prior to administration of the TAT-FXN fusion protein. In some embodiments, the he level of FXN protein in a buccal tissue sample following administration of the TAT-FXN fusion protein for at least 90 days is increased by about 0.5 to about 2.6 pg/μg of total protein, as compared to the level of FXN protein prior to administration of the TAT-FXN fusion protein.

In some embodiments, the level of FXN protein in a buccal tissue sample following administration of the TAT-FXN fusion protein is increased to at least about 30% of the level of FXN protein in a healthy subject following administration of the TAT-FXN fusion protein for at least 30 days, at least 60 days, or at least 90 days.

In some embodiments, the level of FXN protein in a skin biopsy sample following administration of the TAT-FXN fusion protein for at least 30 days ranges from about 4.5 to about 8.5 pg/μg of total protein. In some embodiments, the level of FXN protein in a skin biopsy sample following administration of the TAT-FXN fusion protein for at least 90 days ranges from about 7.0 to about 10.5 pg/μg of total protein.

In some embodiments, the level of FXN protein in a skin biopsy sample following administration of the TAT-FXN fusion protein for at least 30 days is increased by about 1.5 to about 6.0 pg/μg of total protein, as compared to the level of FXN protein prior to administration of the TAT-FXN fusion protein. In some embodiments, the level of FXN protein in a skin biopsy sample following administration of the TAT-FXN fusion protein for at least 90 days is increased by about 4.2 to about 9.0 pg/μg of total protein, as compared to the level of FXN protein prior to administration of the TAT-FXN fusion protein.

In some embodiments, the level of FXN protein in a skin biopsy sample following administration of the TAT-FXN fusion protein is increased to at least about 45% of the level of FXN protein in a healthy subject following administration of the TAT-FXN fusion protein for at least 30 days, or increased to at least about 70% of the level of FXN protein in a healthy subject following administration of the TAT-FXN fusion protein for at least 90 days.

In some aspects, the present disclosure provides a method of increasing level of frataxin (FXN) protein in an FXN deficient subject, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 20% as compared to the level of FXN protein prior to the administration of the TAT-FXN fusion protein.

In some embodiments, the solid tissue sample is a skin biopsy. In some embodiments, the solid tissue sample is a buccal tissue.

In some embodiments, the TAT-FXN fusion protein is administered to the subject for at least 3 weeks, at least 4 weeks, at least 5 weeks, at least 6 weeks, at least 7 weeks, at least 8 weeks, at least 9 weeks, at least 10 weeks, or at least 12 weeks. In some embodiments, the TAT-FXN fusion protein is administered to the subject for more than 10 weeks. In some embodiments, the level of FXN protein in a solid tissue sample obtained from the subject following administration of the TAT-FXN fusion protein is increased by at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%. In some embodiments, the level of FXN protein in a solid tissue sample obtained from the subject following administration of the TAT-FXN fusion protein is increased to at least about 25%, at least about 37.5%, at least about 50%, or at least about 70% of the level of FXN protein in a healthy subject.

In some aspects, the present disclosure provides a method of treating Friedreich's Ataxia in a subject in need thereof, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 20% as compared to the level of FXN protein prior to the administration of the TAT-FXN fusion protein.

In some embodiments, the solid tissue sample is a skin biopsy. In some embodiments, the solid tissue sample is a buccal tissue.

In some embodiments, the TAT-FXN fusion protein is administered to the subject for at least 3 weeks, at least 4 weeks, at least 5 weeks, at least 6 weeks, at least 7 weeks, at least 8 weeks, at least 9 weeks, at least 10 weeks, or at least 12 weeks. In some embodiments, the TAT-FXN fusion protein is administered to the subject for more than 10 weeks.

In some embodiments, the level of FXN protein in a solid tissue sample obtained from the subject following administration of the TAT-FXN fusion protein is increased by at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%. In some embodiments, the level of FXN protein in a solid tissue sample obtained from the subject following administration of the TAT-FXN fusion protein is increased to at least about 25%, at least about 37.5%, at least about 50%, or at least about 70% of the level of FXN protein in a healthy subject.

### BRIEF DESCRIPTION OF THE DRAWINGS

FIG. 1 is a schematic showing administration schedule 25 mg and 50 mg doses of the TAT-FXN fusion protein or placebo to subjects with FRDA in the dose exploration study.

FIG. 2 is a graph showing dose-dependent increase from baseline in FXN levels in skin cells of subjects with FRDA that were administered 25 mg or 50 mg of the TAT-FXN fusion protein or placebo at day 14 and day 28. The dots represent median and the horizontal lines represent 25th and 75th percentiles.

FIG. 3 is a waterfall plot showing the individual changes in FXN levels from baseline in skin cells of subjects with FRDA who were administered 25 mg or 50 mg of the TAT-FXN fusion protein or placebo at day 14.

FIG. 4 is a graph showing dose-dependent increase from baseline in FXN levels in buccal cells of subjects with FRDA who were administered 25 mg or 50 mg of the TAT-FXN fusion protein or placebo at day 14 and day 28. The dots represent median and the horizontal lines represent 25th and 75th percentiles.

FIG. 5 is a waterfall plot showing the individual changes in FXN levels from baseline in buccal cells of subjects with FRDA that were administered 25 mg or 50 mg of the TAT-FXN fusion protein or placebo at day 14.

FIG. 6 is a schematic showing the number of subjects who had a shift in their FXN levels in skin cells as a percentage of healthy volunteers after 14 days of treatment with the TAT-FXN fusion protein.

FIG. 7 is a schematic showing the number of subjects who had a shift in their FXN levels in buccal cells as a percentage of healthy volunteers after 14 days of treatment with the TAT-FXN fusion protein.

FIG. 8 is a schematic showing administration schedules of 25 mg, 50 mg and 100 mg doses of TAT-FXN fusion protein or placebo to subjects with FRDA in the multiple ascending dose study.

FIG. 9 is a diagram showing the PK/PD model developed in the study described in Example 2. Model parameters were Kin (zero order rate constant for FXN) 0.0241 1/h; EBASE (estimated baseline) 3.28 pg/μgₚᵣₒₜ; F4 (proportionality constant for FXN input from central compartment) 0.0194 103 pg/μg/μgₚᵣₒₜ.

FIG. 10 is a plot showing mean and (SD) PK parameters for doses of 25 mg, 50 mg and 100 mg for the last day of daily dose for the multiple ascending dose study.

FIG. 11 is a plot showing mean and SD PK parameters with doses of 25 mg and 50 mg for the last day of daily dose for the dose exploration study.

FIG. 12 is a plot showing skin frataxin to protein ratio over time predicted from PK/PD simulations for a dose of 100 mg, 75 mg, 50 mg and 25 mg of the TAT-FXN fusion protein. Dashed red line represents 50% of the average skin frataxin/protein ratio (8.17 pg/μg) in a non-interventional study in healthy controls. Blue line represents the median of simulated values across trials, and the red lines are the 10th and 90th percentiles. Shaded regions represent the 95% confidence intervals of the corresponding percentiles (10th, 50th, and 90th).

FIG. 13 is a plot showing model-predicted maximum skin FXN concentration at steady state stratified by baseline FXN level. The black dashed lines represent 50% (8.17 pg/mcg) and 100% (16.35 pg/mcg) of the mean skin FXN concentrations in healthy controls. The densities correspond to the 10th percentile (yellow), medians (green), and 90th percentile (red) of the simulated maximum skin FXN to protein ratio at steady state.

FIG. 14 is a plot showing model-predicted maximum skin FXN concentration at steady state stratified by age of onset. The black dashed lines represent 50% (8.17 pg/mcg) and 100% (16.35 pg/mcg) of the mean skin FXN concentrations in healthy controls. The densities correspond to the 10th percentile (yellow), medians (green), and 90th percentile (red) of the simulated maximum skin FXN to protein ratio at steady state.

FIG. 15 is a graph showing the relationship between age at onset and GAA repeat length in adults with FRDA participating in interventional clinical studies with the TAT-FXN fusion protein.

FIG. 16 is a graph showing the relationship between buccal cell and skin cell frataxin concentrations in adults with FRDA participating in interventional clinical studies with the TAT-FXN fusion protein.

FIG. 17 is a graph showing the relationship between buccal cell frataxin concentration and age at symptom onset in adults with FRDA participating in interventional clinical studies with TAT-FXN fusion protein.

FIG. 18 is a graph showing the relationship between buccal cell frataxin concentration and shorter triplet GAA (guanine adenine adenine) repeat lengths in adults with FRDA participating in interventional clinical studies with the TAT-FXN fusion protein.

FIG. 19 is a graph showing the relationship between buccal cell frataxin concentration and total modified Freidreich's ataxia rating scale (mFARS) score in adults with FRDA participating in interventional clinical studies with the TAT-FXN fusion protein.

FIG. 20 is a graph showing frataxin levels (pg/μg of total protein) in skin cells measured at baseline, at Day 30 and Day 90 following daily administration of 25 mg of CTI-1601. The dotted line indicates frataxin levels that are 50% of the frataxin levels in healthy volunteers.

FIG. 21 is a graph showing change from baseline in frataxin levels (pg/μg of total protein) in skin cells measured at Day 30 and Day 90 following daily administration of 25 mg of CTI-1601.

FIG. 22 is a graph showing frataxin levels (pg/μg of total protein) in buccal cells measured at baseline, at Day 30, Day 60 and Day 90 following daily administration of 25 mg of CTI-1601. The dotted line indicates frataxin levels that are 50% of the frataxin levels in healthy volunteers.

FIG. 23 is a graph showing change from baseline in frataxin levels (pg/μg of total protein) in buccal cells measured at Day 30, Day 60 and Day 90 following daily administration of 25 mg of CTI-1601.

FIG. 24 shows increase in frataxin levels in buccal cells as a percentage of average frataxin levels in healthy volunteers at baseline, Day 30, Day 60 and Day 90 following daily administration of 25 mg of CTI-1601.

FIG. 25 shows increase in frataxin levels in skin cells as a percentage of average frataxin levels in healthy volunteers at baseline, Day 30 and Day 90 following daily administration of 25 mg of CTI-1601.

FIG. 26 is a schematic showing the number of subjects who had a shift in their frataxin levels in buccal cells as a percentage of frataxin levels of healthy volunteers following 30 days, 60 days or 90 days of daily dosing with the TAT-FXN fusion protein.

FIG. 27 is a schematic showing the number of subjects who had a shift in their frataxin levels in skin cells as a percentage of frataxin levels of healthy volunteers following 30 days, or 90 days of daily dosing with the TAT-FXN fusion protein.

### DETAILED DESCRIPTION OF THE DISCLOSURE

The present disclosure provides a method of increasing level of frataxin (FXN) protein in an FXN deficient subject, the method comprising administering to the subject a TAT-FXN fusion protein at a dose of about 25 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased, as compared to the level of FXN protein prior to the administration of the TAT-FXN fusion protein.

The present disclosure also provides a method of increasing level of frataxin (FXN) protein in an FXN deficient subject, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 20%, as compared to the level of FXN protein prior to the administration of the TAT-FXN fusion protein.

The present disclosure also provides a method of increasing level of frataxin (FXN) protein in an FXN deficient subject, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 1.2-fold, as compared to the level of FXN protein prior to the administration of the TAT-FXN fusion protein.

The present disclosure also provides a method of increasing level of frataxin (FXN) protein in an FXN deficient subject, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 3 pg/μg of total protein, as compared to the level of FXN protein prior to the administration of the TAT-FXN fusion protein.

The present disclosure also provides a method of treating Friedreich's Ataxia (FRDA) in a subject in need thereof, the method comprising administering to the subject a TAT-FXN fusion protein at a dose of about 25 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased, as compared to the level of FXN protein prior to the administration of the TAT-FXN fusion protein.

The present disclosure also provides a method of treating Friedreich's Ataxia in a subject in need thereof, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 20%, as compared to the level of FXN protein prior to the administration of the TAT-FXN fusion protein.

The present disclosure also provides a method of treating Friedreich's Ataxia in a subject in need thereof, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least 1.2-fold, as compared to the level of FXN protein prior to the administration of the TAT-FXN fusion protein.

The present disclosure also provides a method of treating Friedreich's Ataxia in a subject in need thereof, the method comprising administering to the subject a TAT-FXN fusion protein at a dose of about 50 mg; wherein the dose is administered once daily for at least 14 days; and wherein the TAT-FXN fusion protein comprises the amino acid sequence set forth in SEQ ID NO: 5; such that the level of FXN protein in a solid tissue sample obtained from the subject following administration of the TAT-FXN fusion protein is increased by at least about 3 pg/μg of total protein, as compared to the level of FXN protein prior to the administration of the TAT-FXN fusion protein.

In some embodiments, when the subject is administered the TAT-FXN fusion protein once daily at a dose of about 25 mg, the solid tissue sample is obtained from the subject at least 14 days following the start of the administration of the TAT-FXN fusion protein. In some embodiments, the solid tissue sample is obtained from the subject at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 25 weeks, at least 30 weeks, at least 35 weeks, at least 40 weeks, at least 45 weeks, at least 50 weeks or at least 60 or more weeks following the start of administration of the FXN fusion protein.

In some embodiments, when the subject is administered the TAT-FXN fusion protein once daily at a dose of about 50 mg, the solid tissue sample is obtained from the subject at least 14 days following the start of the administration of the TAT-FXN fusion protein. In some embodiments, the solid tissue sample is obtained from the subject at least at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 25 weeks, at least 30 weeks, at least 35 weeks, at least 40 weeks, at least 45 weeks, at least 50 weeks or at least 60 or more weeks following the start of administration of the FXN fusion protein.

In some embodiments, the solid tissue sample is obtained at more than one time point, e.g., at 14 days and at 28 days, following the start of administration of the TAT-FXN fusion protein. In some embodiments, the level of FXN in the tissue sample is determined at more than one time point following the start of administration of the TAT-FXN fusion protein (e.g., once every week, once every 2 weeks, once every 3 weeks, or once a month) in order to monitor the treatment of the subject with the TAT-FXN fusion protein.

In some embodiments, the methods further comprise the step of obtaining the solid tissue sample or samples from the subject. In some embodiments, the methods further comprise the step of measuring or determining the concentration of FXN in the solid tissue sample or samples obtained from the subject. In some embodiments, the methods further comprise the step of receiving information on the concentration of FXN in the solid tissue sample or samples obtained from the subject.

In some embodiments, the methods further comprise making a decision on whether to maintain or to modify a dose amount or dosing frequency based on the concentration of FXN in the solid tissue sample or samples. In some embodiments, the methods further comprise making a recommendation on whether to maintain or to modify a dose amount or dosing frequency based on the concentration of FXN in the solid tissue sample or samples.

In some embodiments, the subject is administered the TAT-FXN fusion protein once daily at a dose of about 25 mg for at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 25 weeks, at least 30 weeks, at least 35 weeks, at least 40 weeks, at least 45 weeks, at least 50 weeks or at least 60 or more weeks.

In some embodiments, the subject is administered the TAT-FXN fusion protein once daily for at least 30 days, at least 60 days or at least 90 days.

In some embodiments, the level of FXN protein in a solid tissue sample obtained from a subject who is administered the TAT-FXN fusion protein once daily at a dose of about 25 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 10%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, at least about 1000%, at least about 1500%, at least about 2000%, at least about 2500%, at least about 5000% or at least about 10000%. In some embodiments, the solid tissue sample is a skin sample. In some embodiments, the solid tissue sample is a buccal sample.

In some embodiments, the level of FXN protein in a solid tissue sample obtained from a subject who is administered the TAT-FXN fusion protein once daily at a dose of about 25 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 500-fold or at least about 1000-fold. In some embodiments, the solid tissue sample is a skin sample. In some embodiments, the solid tissue sample is a buccal sample.

In some embodiments, the level of FXN protein in a solid tissue sample obtained from a subject who is administered the TAT-FXN fusion protein once daily at a dose of about 25 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 1 pg/μg of total protein, at least about 2 pg/μg of total protein, at least about 3 pg/μg of total protein, at least about 4 pg/μg of total protein, at least about 5 pg/μg of total protein, at least about 6 pg/μg of total protein, at least about 7 pg/μg of total protein, at least about 8 pg/μg of total protein, or at least about 9 pg/μg of total protein. In some embodiments, the solid tissue sample is a skin sample. In some embodiments, the solid tissue sample is a buccal sample.

In some embodiments, the level of FXN protein in a buccal tissue sample following administration of the TAT-FXN fusion protein at a daily dose of about 25 mg for at least 30 days is about 0.5 to about 3.0 pg/μg of total protein.

In some embodiments, the level of FXN protein in a buccal tissue sample following administration of the TAT-FXN fusion protein at a daily dose of about 25 mg for at least 60 days is about 1.0 to about 3.3 pg/μg of total protein.

In some embodiments, the level of FXN protein in a buccal sample following administration of the TAT-FXN fusion protein at a daily dose of about 25 mg for at least 90 days is about 1.4 to about 3.7 pg/μg of total protein.

In some embodiments, the level of FXN protein in a skin biopsy sample following administration of the TAT-FXN fusion protein at a daily dose of about 25 mg for at least 30 days is about 4.5 to about 8.5 pg/μg of total protein.

In some embodiments, the level of FXN protein in a skin biopsy sample following administration of the TAT-FXN fusion protein at a daily dose of about 25 mg for at least 90 days is about 7.0 to about 10.5 pg/μg of total protein.

In some embodiments, the level of FXN protein in a solid tissue sample obtained from a subject who is administered the TAT-FXN fusion protein once daily at a dose of about 25 mg is increased to at least about 12.5%, at least about 25%, at least about 37.5%, at least about 50% or at least about 70% of the level of FXN in the solid tissue sample of a healthy subject. The term “level of FXN protein in a healthy subject” or “level of FXN protein in the solid tissue sample of a healthy subject”, as used herein, refers to the level of FXN protein in a solid tissue sample (e.g., buccal or skin sample) from one or more healthy subjects, e.g., an average (e.g., a mean or median) of FXN levels in a solid tissue sample (e.g., buccal or skin sample) from multiple (e.g., 2, 3, 4, 5, 10, 15, 20 or more) healthy subjects. In some embodiments, the solid tissue sample is a skin sample. In some embodiments, the level of FXN protein in a healthy subject is about 10 pg/μg of total protein to about 20 pg/μg of total protein, or about 14 pg/μg of total protein to about 18 pg/μg of total protein, or about 15 pg/μg of total protein to about 17 pg/μg of total protein, e.g., about 16.34 pg/μg of total protein in a skin biopsy sample. In some embodiments, the solid tissue sample is a buccal sample. In some embodiments, the level of FXN protein in a healthy subject is about 5 pg/μg of total protein to about 12 pg/μg of total protein, about 6 pg/μg of total protein to about 10 pg/μg of total protein, or about 7 pg/μg of total protein to about 9 pg/μg of total protein, e.g., about 8.24 pg/μg of total protein, in a buccal sample.

In some embodiments, the level of FXN protein in a buccal tissue sample is increased to at least about 30% of the level of FXN protein in a healthy subject following administration of the TAT-FXN fusion protein at a daily dose of about 25 mg for at least 30 days, at least 60 days, or at least 90 days.

In some embodiments, the level of FXN protein in a buccal tissue sample following administration of the TAT-FXN fusion protein at a daily dose of about 25 mg for at least 30 days is increased by about 0.01 to about 1.5 pg/μg of total protein, as compared to the level of FXN protein prior to administration of the TAT-FXN fusion protein.

In some embodiments, the level of FXN protein in a buccal tissue sample following administration of the TAT-FXN fusion protein at a daily dose of about 25 mg for at least 60 days is increased by about 0.10 to about 1.8 pg/μg of total protein, as compared to the level of FXN protein prior to administration of the TAT-FXN fusion protein.

In some embodiments, the level of FXN protein in a buccal tissue sample following administration of the TAT-FXN fusion protein at a dose of about 25 mg for at least 90 days is increased by about 0.5 to about 2.6 pg/μg of total protein, as compared to the level of FXN protein prior to administration of the TAT-FXN fusion protein.

In some embodiments, the level of FXN protein in a skin biopsy sample following administration of the TAT-FXN fusion protein is increased to at least about 45% of the level of FXN protein in a healthy subject following administration of the TAT-FXN fusion protein for at least 30 days, or increased to at least about 70% of the level of FXN protein in a healthy subject following administration of the TAT-FXN fusion protein at a daily dose of about 25 mg for at least 90 days.

In some embodiments, the level of FXN protein in a skin biopsy sample following administration of the TAT-FXN fusion protein at a daily dose of about 25 mg for at least 30 days is increased by about 1.5 to about 6.0 pg/μg of total protein, as compared to the level of FXN protein prior to administration of the TAT-FXN fusion protein.

In some embodiments, the level of FXN protein in a skin biopsy sample following administration of the TAT-FXN fusion protein at a daily dose of about 25 mg for at least 90 days is increased by about 4.2 to about 9.0 pg/μg of total protein, as compared to the level of FXN protein prior to administration of the TAT-FXN fusion protein.

In some embodiments, the increase in the level of hFXN in the solid tissue sample obtained from the subject, e.g., a skin sample or a buccal sample, is sufficient to have a therapeutic effect, i.e., sufficient to treat FRDA in the subject.

In some embodiments, the subject is administered the TAT-FXN fusion protein once daily at a dose of about 50 mg for at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 25 weeks, at least 30 weeks, at least 35 weeks, at least 40 weeks, at least 45 weeks, at least 50 weeks or at least 60 or more weeks.

In some embodiments, the level of FXN protein in a solid tissue sample obtained from a subject who is administered the TAT-FXN fusion protein once daily at a dose of about 50 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 25%, at least about 30%, at least about 35%, at least about 40%, at least about 45%, at least about 50%, at least about 55%, at least about 60%, at least about 70%, at least about 80%, at least about 90%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, at least about 1000%, at least about 1500%, at least about 2000%, at least about 2500%, at least about 5000% or at least about 10000%. In some embodiments, the solid tissue sample is a skin sample. In some embodiments, the solid tissue sample is a buccal sample.

In some embodiments, the level of FXN protein in a solid tissue sample obtained from a subject who is administered the TAT-FXN fusion protein once daily at a dose of about 50 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 500-fold or at least about 1000-fold. In some embodiments, the solid tissue sample is a skin sample. In some embodiments, the solid tissue sample is a buccal sample.

In some embodiments, the level of FXN protein in a solid tissue sample obtained from a subject who is administered the TAT-FXN fusion protein once daily at a dose of about 50 mg is increased following administration of the TAT-FXN fusion protein, as compared to the level of FXN protein in a solid sample obtained from the subject prior to the administration of the TAT-FXN fusion protein. In some embodiments the level of FXN protein is increased by at least about 1 pg/μg of total protein, at least about 2 pg/μg of total protein, at least about 3 pg/μg of total protein, at least about 4 pg/μg of total protein, at least about 5 pg/μg of total protein, at least about 6 pg/μg of total protein, at least about 7 pg/μg of total protein, at least about 8 pg/μg of total protein, or at least about 9 pg/μg of total protein. In some embodiments, the solid tissue sample is a skin sample. In some embodiments, the solid tissue sample is a buccal sample.

In some embodiments, the level of FXN protein in a solid tissue sample obtained from a subject who is administered the TAT-FXN fusion protein once daily at a dose of about 50 mg is increased to at least about 12.5%, at least about 25%, at least about 37.5% or at least about 50% of the level of FXN in the solid tissue sample of a healthy subject. In some embodiments, the level of FXN in a solid tissue sample may be an average or a median of FXN levels measured in a solid tissue sample of multiple, e.g., two or more, subjects. In some embodiments, the solid tissue sample is a buccal sample.

In some embodiments, the increase in the level of hFXN in the solid tissue sample obtained from the subject, e.g., a skin sample or a buccal sample, is sufficient to have a therapeutic effect, i.e., sufficient to treat FRDA in the subject.

The term “FXN protein”, as used herein, encompasses both the full-length human frataxin protein (hFXN) and mature hFXN. The full-length hFXN contains 210 amino acids, and includes an 80 amino acid mitochondrial targeting sequence (MTS) at the N-terminus. The full-length hFXN protein (amino acids 1-210) has the amino acid sequence of SEQ ID NO: 1.

| | |
|---|---|
| SEQ ID NO: 1 | MWTLGRRAVAGLLASPSPAQAQTLTRVPRPAELAPLCG |
| Full-length | RRGLRTDIDATCTPRRASSNQRGLNQIWNVKKQSVYLM |
| hFXN | NLRKSGTLGHPGSLDETTYERLAEETLDSLAEFFEDLA |
| hFXN₁₋₂₁₀ | DKPYTFEDYDVSFGSGVLTVKLGGDLGTYVINKQTPNK |
| | QIWLSSPSSGPKRYDWTGKNWVYSHDGVSLHELLAAEL |
| | TKALKTKLDLSSLAYSGKDA |

As the hFXN protein is imported into the mitochondrial matrix, it is cleaved at amino acid 81, resulting in the mature form of FXN, having 130 aa and a predicted molecular weight of 14.2 kDa (SEQ ID NO: 2).

| | |
|---|---|
| SEQ ID NO: 2 | SGTLGHPGSLDETTYERLAEETLDSLAEFFEDLADKPY |
| Mature hFXN | TFEDYDVSFGSGVLTVKLGGDLGTYVINKQTPNKQIWL |
| hFXN₈₁₋₂₁₀ | SSPSSGPKRYDWTGKNWVYSHDGVSLHELLAAELTKAL |
| | KTKLDLSSLAYSGKDA |

The full-length hFXN (SEQ ID NO: 1) comprises mature hFXN (SEQ ID NO: 2) and a mitochondrial targeting sequence (MTS) having the amino acid sequence
MWTLGRRAVAGLLASPSPAQAQTLTRVPRPAELAPLCGRRGLRTDIDATCTPRRASSNQ RGLNQIWNVKKQSVYLMNLRK (SEQ ID NO: 3).

The TAT-FXN fusion protein of the present disclosure includes hFXN and also the HIV-TAT peptide YGRKKRRQRRR (SEQ ID NO: 4) linked via a linker to the N-terminus of the full-length hFXN protein. The mechanism of action of the fusion protein relies on the cell-penetrating ability of the HIV-TAT peptide to deliver the fusion protein into cells and the subsequent processing into mature hFXN after translocation into the mitochondria. The TAT-FXN fusion protein of the present disclosure, which may also be referred to herein as “CTI-1601” has the following amino acid sequence (224 amino acids):

(SEQ ID NO: 5)
MYGRKKRRQRRRGGMWTLGRRAVAGLLASPSPAQAQTLTRVPRPAELAPL
CGRRGLRTDIDATCTPRRASSNQRGLNQIWNVKKQSVYLMNLRKSGTLGH
PGSLDETTYERLAEETLDSLAEFFEDLADKPYTFEDYDVSFGSGVLTVKL
GGDLGTYVINKQTPNKQIWLSSPSSGPKRYDWTGKNWVYSHDGVSLHELL
AAELTKALKTKLDLSSLAYSGKDA.

In some embodiments, the TAT-FXN fusion protein of the present disclosure comprises an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 5.

In some embodiments, the TAT-FXN fusion protein of the present disclosure is a protein consists of an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 5.

In some embodiments, the subject is a human subject who is at least 18 years old. In other embodiments, the subject is a human subject who is less than 18 years old.

In some embodiments, the TAT-FXN fusion protein is administered subcutaneously.

In some embodiments, the TAT-FXN fusion protein is administered as a part of a pharmaceutical composition comprising the TAT-FXN fusion protein, a pharmaceutically acceptable excipient and a pharmaceutically acceptable carrier. An exemplary pharmaceutical composition comprising the TAT-FXN fusion protein is described, e.g., in WO 2022/126029, the entire contents of which are hereby incorporated herein by reference.

In some embodiments, the levels of FXN protein are measured using mass spectrometry, e.g., a combination of immunopurification and mass spectrometry. An exemplary method of measuring levels of FXN protein using mass spectrometry comprises detecting a peptide derived from mature hFXN, e.g., a peptide comprising or consisting of the amino acid sequence of SGTLGHPGSLDETTYER (SEQ ID NO: 6); and/or a peptide comprising, or consisting of, the amino acid sequence LGGDLGTYVINK (SEQ ID NO: 7). An exemplary method that may be used to measure levels of FXN protein in a solid sample obtained from a subject is described, e.g., in WO 2021/021964, the entire contents of which are hereby incorporated herein by reference. An exemplary method of measuring levels of FXN protein may comprise using liquid chromatography, followed by tandem mass spectrometry (LC/MS-MS) and monitoring the transition 607.3→669.3 for the peptide comprising, or consisting of, SEQ ID NO: 6 and the transition 625.3→794.4 for the peptide comprising, or consisting of, SEQ ID NO: 7. In some embodiments, an exemplary method of measuring levels of FXN protein in a solid tissue sample may comprise (a) purifying by immunocapture the FXN protein from the solid tissue sample, thereby obtaining immunocaptured complexes comprising FXN; (b) subjecting the immunocaptured complexes comprising FXN to digestion by trypsin, thereby producing the peptide comprising SEQ ID NO: 6 and/or the peptide comprising SEQ ID NO: 7; and (c) determining the amount of at least one peptide selected from the group consisting of the peptide comprising SEQ ID NO: 6 and the peptide comprising SEQ ID NO: 7 by LC/MS-MS.

### Treatment of Friedreich's Ataxia

In the context of the present disclosure, administration of a TAT-FXN fusion protein at a dose of between 25 and 50 mg, e.g., a dose of about 25 mg for at least 14 days, or at a dose of about 50 mg for at least 14 days, can be clinically effective to treat Friedreich's ataxia (FRDA).

It is presently anticipated that protein replacement therapy with the TAT-FXN fusion protein will correct the metabolic defect in FRDA and restore adequate cellar function in subjects. It is also anticipated that treatment with the TAT-FXN fusion protein will change FRDA from a progressive and deadly disease to a chronic condition that is managed by injections of the fusion polypeptide, much as insulin has changed diabetes into a chronic disease with normal life activities. In older FRDA patients with established disease, it is anticipated that administration of the TAT-FXN fusion protein will halt disease progression. In children diagnosed before onset of FRDA symptoms, it is anticipated that administration of the TAT-FXN fusion protein will result in near complete preservation of tissue function and health.

The gene defect for FRDA was identified in 1996 and there is consensus in the field that lack of FXN protein in mitochondria is the biochemical defect. Multiple investigators have shown that replacement of FXN in deficient patient fibroblasts, and even in yeast with loss of FXN, will rescue the phenotype. Thus, the consensus in the field is that therapies for FRDA must include increasing levels of FXN protein in mitochondria of affected tissues. Although the precise function of FXN has yet to be defined, it is clear that FXN participates in iron-sulfur cluster assembly. In its absence, mitochondrial proteins containing an iron-sulfur cluster (Complexes I, II, and III of the electron transport chain, and aconitase of the Krebs cycle) are severely defective in activity. As a result, those tissues with high dependence on energy production by mitochondria, such as heart and brain, are severely affected and greater than about 60% of patients die from heart failure. As with other mitochondrial diseases, multiple organ systems are also impacted, such as eye, hearing, and pancreas. Thus, clinically relevant target tissues include the heart and brain and can be followed by common clinical testing, such as echocardiography, and neurologic assays such as the Friedreich Ataxia Rating Scale (FARS).

Administration of a TAT-FXN fusion protein, e.g., at a dose of about 25 mg administered once daily for at least 14 days, or at a dose of 50 mg administered once daily for at least 14 days, can, therefore, be effective as a protein replacement therapy in FXN-deficient subjects diagnosed with FRDA, including, e.g., an FRDA-associated disease, disorder or condition, to treat the FRDA, including, e.g., the FRDA-associated disease, disorder or condition.

The term “FRDA”, as used herein, encompasses any disease, disorder or condition associated with a frataxin deficiency. In some embodiments, “FRDA” may be characterized by a loss of function in FXN, e.g., a loss of function mutation in an FXN gene. The term “FRDA-associated disease, disorder or condition”, as used herein, encompasses a disease, disorder or condition secondary to and/or caused by FRDA, i.e., when present in a subject, it accompanies FRDA and is not present in a subject in the absence of FRDA. Non-limiting examples of an FRDA-associated disease, disorder, or condition, include FRDA-associated pneumonia, FRDA-associated hypertrophic cardiomyopathy and FRDA-associated diabetes. Other non-limiting examples of an FRDA-associated disease, disorder or condition include an FRDA-associated disease, disorder or condition characterized by, without limitation:
(1) a neurological deficiency including, without limitation, one or more of the following: loss of proprioception, loss of reflexes, loss of ability to walk, loss of ability to hold gaze with eyes;
(2) impaired swallowing and/or a progressive loss of the ability to swallow; progressive loss of hearing;
(3) progressive loss of vision due to retinal degeneration from lack of FXN;
(4) progressive loss of speech;
(5) metabolic syndrome including, without limitation, elevated triglycerides, low high-density lipoprotein (HDL) cholesterol, and elevated low-density lipoprotein (LDL) cholesterol;
(6) scoliosis that requires surgery to correct; and/or combinations thereof.

In some embodiments, administration of a TAT-FXN fusion protein, e.g., at a dose of about 25 mg administered once daily for at least 14 days, or at a dose of 50 mg administered once daily for at least 14 days, to a subject treats FRDA, including, e.g., an FRDA-associated disease, disorder or condition. “Treating FRDA”, as used herein, encompasses ameliorating, improving or achieving a reduction in the severity of FRDA, including, e.g., an FRDA-associated disease, disorder or condition. For example, “treating FRDA” encompasses ameliorating, improving or achieving a reduction in at least one symptom or indicator associated with FRDA. “Treating FRDA”, as used herein, also encompasses delaying progression of FRDA, including, e.g., an FRDA-associated disease disorder or condition, e.g., delaying appearance of at least one symptom or indicator associated with FRDA or preventing an increase in the severity of at least one symptom or indicator associated with FRDA, in a subject.

In some embodiments, the term “treating FRDA” also encompasses achieving increased survival (e.g., survival time) of a subject, e.g., a human, with FRDA, including, e.g., an FRDA-associated disease, disorder or condition. For example, treatment of FRDA may result in an increased life expectancy of a subject, e.g., a human, with FRDA, including, e.g., an FRDA-associated disease disorder or condition. In some embodiments, treatment of FRDA in the context of the present disclosure may result in an increased life expectancy of a subject of greater than about 10%, greater than about 20%, greater than about 30%, greater than about 40%, greater than about 50%, greater than about 60%, greater than about 70%, greater than about 80%, greater than about 90%, greater than about 100%, greater than about 110%, greater than about 120%, greater than about 130%, greater than about 140%, greater than about 150%, greater than about 160%, greater than about 170%, greater than about 180%, greater than about 190%, or greater than about 200% or more, as compared to the average life expectancy of one or more control individuals with similar disease without treatment.

In some embodiments, treatment of FRDA, including, e.g., an FRDA-associated disease, disorder or condition, in the context of the present disclosure may result in an increased life expectancy of a subject by greater than about 6 months, greater than about 8 months, greater than about 10 months, greater than about 12 months, greater than about 2 years, greater than about 4 years, greater than about 6 years, greater than about 8 years, or greater than about 10 years or more, as compared to the average life expectancy of one or more control individuals with similar disease without treatment. In some embodiments, treatment of FRDA, including, e.g., an FRDA-associated disease, disorder or condition in the context of the present disclosure may result in a long-term survival of a subject, e.g., a human, with FRDA, including, e.g., an FRDA-associated disease, disorder or condition. The term “long-term survival”, as used herein, refers to a survival time or life expectancy longer than about 40 years, 45 years, 50 years, 55 years, 60 years, or longer.

Clinical assessments known to one of ordinary skill in the art may be used to assess FRDA, including, e.g., an FRDA-associated disease, disorder or condition, to determine the severity of the FRDA and/or to determine the effect of administration to a subject of a TAT-FXN fusion protein at a dose of about 25 mg once daily for at least 14 days or a dose of about 50 mg once daily for at least 14 days. Examples of methods of clinical assessment of FRDA, including assessments of the severity of FRDA, are described, e.g., in Paap et al., “Standardized Assessment of Hereditary Ataxia Patients in Clinical Studies”, Mov Disord Clin Pract. 2016, 3 (3): 230-240 and Patel et al., “Progression of Friedreich ataxia: quantitative characterization over 5 years”, Ann Clin Transl Neurol 2016, 3 (9): 684-694, the entire contents of each of which are hereby incorporated herein by reference.

Timed 25-Foot Walk (T25-FW) is a quantitative mobility and leg function performance test that measures the time needed to complete a 25-foot walk. In some embodiments, administration to a subject of a TAT-FXN fusion protein at a dose of about 25 mg once daily for at least 14 days or a dose of about 50 mg once daily for at least 14 days may result in a decrease in the severity of FRDA as measured, e.g., by the time needed to complete a 25-foot walk. For example, administration to a subject of a TAT-FXN fusion protein at a dose of about 25 mg once daily for at least 14 days or a dose of about 50 mg once daily for at least 14 days may result in a decrease in the time needed to complete a 25-foot walk, e.g., a decrease of at least about 5%, at least about 10%, at least about 25%, or at least about 50% in the time needed to complete a 25-foot walk, as compared to the time needed to complete a 25-foot walk measured in the subject prior to administration of the TAT-FXN fusion protein, or as compared to a baseline value. A baseline value may be the time needed to complete a 25-foot walk measured prior to administration of the TAT-FXN fusion protein.

In other embodiments, administration to a subject of a TAT-FXN fusion protein at a dose of about 25 mg once daily for at least 14 days or a dose of about 50 mg once daily for at least 14 days may delay progression of FRDA in the subject as measured, e.g., by the time needed to complete a 25-foot walk. For example, administration to a subject of a TAT-FXN fusion protein at a dose of about 25 mg once daily for at least 14 days or a dose of about 50 mg once daily for at least 14 days may result in a substantially similar time needed to complete a 25-foot walk, or a lack of a substantial increase in the time needed to complete a 25-foot walk (e.g., less than a 20%, less than a 10%, or less than a 5% increase in the time needed to complete a 25-foot walk), as compared to the baseline value, i.e., time needed to complete a 25-foot walk measured in the subject prior to administration of the TAT-FXN fusion protein.

The Modified Friedreich's Ataxia Rating Scale (mFARS) is an examination-based rating scale for assessing the severity of FRDA as described, e.g., in Burk et al., “Monitoring progression in Friedreich ataxia (FRDA): the use of clinical scales”, J of Neurochemistry 2013, 126 (suppl. 1): 118-124 and Rummey et al., “Psychometric properties of the Friedreich's Ataxia Rating Scale”, Neurol Genet 2019, 5: e371, the entire contents of each of which are hereby incorporated herein by reference.

In some embodiments, the mFARS score may comprise at least one of the following subscores: a) a score based on the Functional Disability Rating Scale (FARS-FDS; 0-6 scale; assessment usually made by a neurologist; b) a score based on the Activities of Daily Living Scale (FARS-ADL, 0-36 scale; assessment made by a patient or caregiver); and c) a score based on the Neurological Rating Scale (FARS-neuro) 0-125 scale; assessment made by a neurologist). In some examples, the FARS_ADL score is a FARS rating scale assessing subject ability to complete ADLs (e.g., speech, cutting food, dressing, and personal hygiene), with scores ranging from 0 to 36 points. The respondent may be the subject; a combination of the subject and family; or a family member, spouse or caregiver for those subjects unable to complete the test.

In some embodiments, the score based on the Neurological Rating Scale may include modified scoring of the neurological rating scale involving direct subject participation and targeting specific areas impacted by FRDA, such as bulbar, upper limb, lower limb, and upright stability (mFARS-neuro, 0-99 scale). The mFARS-neuro excludes subscale D (peripheral nervous system) and the first 2 questions of subscale A (bulbar) from the neurological rating scale of the FARS questionnaire.

In some embodiments, the mFARS score may be based on two subscores derived from the full FARS questionnaire: mFARS-neuro as described above and the FARS_ADL as described above.

In some embodiments, administration to a subject of a TAT-FXN fusion protein at a dose of about 25 mg once daily for at least 14 days or a dose of about 50 mg once daily for at least 14 days may result in a decrease in the severity of FRDA as measured, e.g., by an mFARS score, or at least one mFARS subscore as described herein. For example, administration to a subject of a TAT-FXN fusion protein at a dose of about 25 mg once daily for at least 14 days or a dose of about 50 mg once daily for at least 14 days may result in a decrease in an mFARS score or at least one mFARS subscore, as compared to a baseline value, i.e., the mFARS score or the at least one mFARS subscore measured in the subject prior to administration of the TAT-FXN fusion protein.

In other embodiments, administration to a subject of a TAT-FXN fusion protein at a dose of about 25 mg once daily for at least 14 days or a dose of about 50 mg once daily for at least 14 days may delay progression of FRDA in the subject as measured, e.g., by an mFARS score or at least one mFARS subscore as disclosed herein. For example, administration to a subject of a TAT-FXN fusion protein at a dose of about 25 mg once daily for at least 14 days or a dose of about 50 mg once daily for at least 14 days may result in a substantially similar mFARS score or at least one mFARS subscore, or a substantial lack of an increase in an mFARS score or at least one mFARS subscore, as compared to a baseline value, i.e., the mFARS score or the at least one mFARS subscore measured in the subject prior to administration of the TAT-FXN fusion protein, or as compared to a baseline value.

The Nine-Hole Peg Test (9HPT) may be used to measure finger dexterity in subjects with FRDA. In this test, a subject is asked to take pegs from a container, one by one, and place them into the nine holes on the board as quickly as possible. The subject must then remove the pegs from the holes, one by one, and replace them back into the container. Scores are based on the time taken to complete the test activity, recorded in seconds.

In some embodiments, administration to a subject of a TAT-FXN fusion protein at a dose of about 25 mg once daily for at least 14 days or a dose of about 50 mg once daily for at least 14 days may result in a decrease in the severity of FRDA as measured, e.g., by a 9HPT score. For example, administration to a subject of a TAT-FXN fusion protein at a dose of about 25 mg once daily for at least 14 days or a dose of about 50 mg once daily for at least 14 days may result in an decrease in a 9HPT score expressed as time to complete the test activity (e.g., at least an about 5%, 10%, 25%, or 50% decrease in a 9HPT score expressed as time to complete the test activity), as compared to a baseline value, i.e., the 9HPT score measured in the subject prior to administration of the TAT-FXN fusion protein.

In other embodiments, administration to a subject of a TAT-FXN fusion protein at a dose of about 25 mg once daily for at least 14 days or a dose of about 50 mg once daily for at least 14 days may delay progression of FRDA in the subject as measured, e.g., by a 9HPT score. For example, administration to a subject of a TAT-FXN fusion protein at a dose of about 25 mg once daily for at least 14 days or a dose of about 50 mg once daily for at least 14 days may result in a substantially similar 9HPT score, or a lack of a substantial increase in a 9HPT score expressed as time to complete the test activity, as compared to a baseline value, i.e., the 9HPT score measured in the subject prior to administration of the TAT-FXN fusion protein.

### Frda-Associated Pneumonia

Subjects diagnosed with FRDA suffer neurodegeneration of the dorsal root ganglia causing progressive ataxia. This typically leads to the progressive loss of an ability to walk, feed oneself, talk, swallow, and pulmonary aspiration. The event of pulmonary aspiration can lead to pneumonia, frequent hospitalizations, and, eventually, death over a period of 10-15 years from the date of diagnosis.

For many of the reasons set forth above, administration of a disclosed pharmaceutical composition comprising a disclosed TAT-FXN fusion protein, can be effective as a protein replacement therapy in FXN-deficient subjects diagnosed with FRDA to prevent pulmonary aspiration, thereby preventing the pneumonia that follows pulmonary aspiration. Accordingly, the present disclosure provides methods of treating an FRDA-associated pneumonia in a subject, comprising administering to a subject in need thereof a pharmaceutical composition comprising a TAT-FXN fusion protein of the disclosure, thereby treating the FRDA-associated pneumonia in the subject.

### FDRA—Associated Hypertrophic Cardiomyopathy

Hypertrophic cardiomyopathy is a condition in which the muscles of the heart thicken, making it difficult for the heart to pump blood through the circulatory system. It can be caused by a deficiency in FXN in the mitochondria of the heart cells. In subjects diagnosed with FRDA, progressive hypertrophic cardiomyopathy about 50% of the time progresses to heart failure and death. Protein replacement therapy with a disclosed TAT-FXN fusion protein can replace the FXN deficiency underlying hypertrophic cardiomyopathy. Administration to a subject of a TAT-FXN fusion protein at a dose of about 25 mg once daily for at least 14 days or a dose of about 50 mg once daily for at least 14 days, can, therefore, be effective as a protein replacement therapy in FXN-deficient subjects diagnosed with both FRDA and hypertrophic cardiomyopathy.

### Diabetes

The hallmark of diabetes is an inability to properly regulate blood levels of glucose, resulting in elevated blood glucose levels. In subjects diagnosed with FRDA, diabetes often shows up as a consequence of FXN-deficient mitochondria in the pancreas. Protein replacement therapy with a TAT-FXN fusion protein can replace the FXN deficiency underlying diabetes. Administration to a subject of a TAT-FXN fusion protein at a dose of about 25 mg once daily for at least 14 days or a dose of about 50 mg once daily for at least 14 days can therefore be effective as a protein replacement therapy in FXN-deficient subjects diagnosed with diabetes.

### Other FRDA-Associated Diseases/Disorders

Subjects diagnosed with FRDA often experience other disorders associated with FXN deficiency. Such FRDA-associated disorders can include, without limitation: neurological disorders including, without limitation, loss of proprioception, loss of reflexes, loss of ability to walk, loss of ability to hold gaze with eyes; impaired swallowing and/or a progressive loss of the ability to swallow; progressive loss of hearing; progressive loss of vision due to retinal degeneration from lack of FXN; progressive loss of speech; metabolic syndrome including, without limitation, elevated triglycerides, low high-density lipoprotein (HDL) cholesterol, and elevated low-density lipoprotein (LDL) cholesterol; scoliosis that requires surgery to correct; and/or combinations thereof. Protein replacement therapy with a disclosed TAT-FXN fusion protein can replace the FXN deficiency underlying these diseases/disorders. Administration to a subject of a TAT-FXN fusion protein at a dose of about 25 mg once daily for at least 14 days or a dose of about 50 mg once daily for at least 14 days, can therefore be effective as a protein replacement therapy in FXN-deficient subjects diagnosed with FRDA and experiencing neurological disorders including, without limitation, loss of proprioception, loss of reflexes, loss of ability to walk, loss of ability to hold gaze with eyes; impaired swallowing and/or a progressive loss of the ability to swallow; progressive loss of hearing; progressive loss of vision due to retinal degeneration from lack of FXN; progressive loss of speech; metabolic syndrome including, without limitation, elevated triglycerides, low HDL cholesterol, and elevated LDL cholesterol; scoliosis that requires surgery to correct; and/or combinations thereof.

### Examples

### Example 1. Administration of TAT-FXN Fusion Protein to Subjects with FRDA Increases Levels of hFXN in Solid Tissues

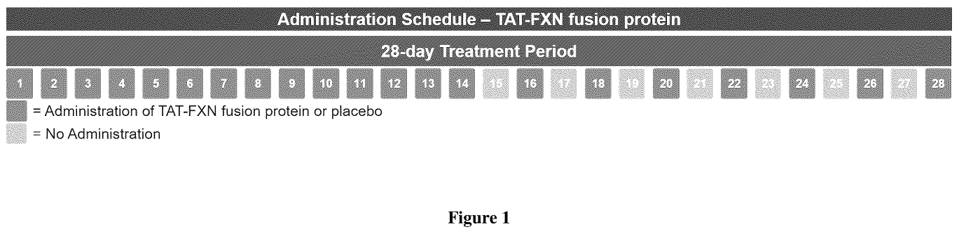

Ambulatory and non-ambulatory subjects with FRDA ≥18 years of age were administered the TAT-FXN fusion protein of SEQ ID NO: 5. Cohort 1 included a total of 13 participants, with 4 participants receiving placebo and 9 participants receiving 25 mg of the TAT-FXN fusion protein. Cohort 2 included a total of 15 participants, with 5 participants receiving placebo and 10 participants receiving 50 mg of the TAT-FXN fusion protein. The TAT-FXN fusion protein was administered once daily for the first 14 days, and once every other day for additional 14 days, as illustrated in FIG. 1.

Skin and buccal samples were collected on day 14 and day 28. FXN levels in the samples were measured using mass spectrometry via detection of peptide derived from mature FXN. FXN concentrations in the samples were determined and normalized to total cellular protein content in each sample. Compared with baseline, median FXN concentration increased by 0.56 pg/μg and 0.72 pg/μg in buccal cells and 2.81 pg/μg and 5.57 pg/μg in skin cells in Cohorts 1 and 2, respectively, after 14 days of daily administration of the TAT-FXN fusion protein, with no change from baseline observed in subjects receiving placebo.

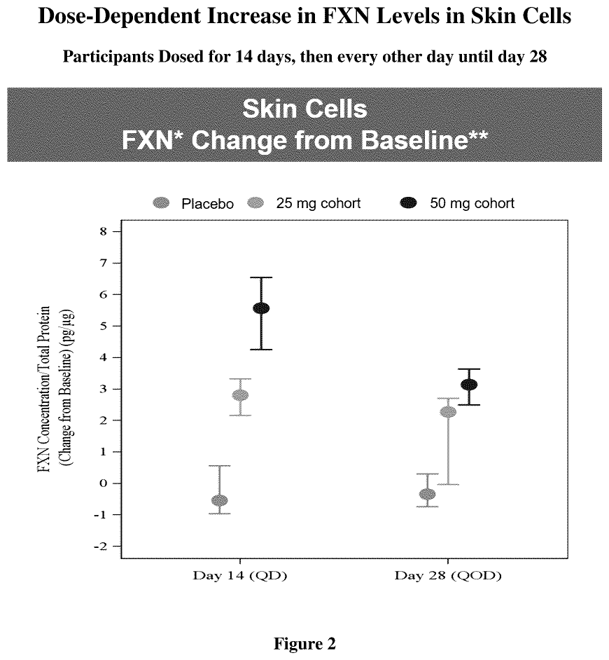

FIG. 2 is a graph showing dose-dependent increase from baseline in FXN levels in skin cells of subjects with FRDA that were administered 25 mg or 50 mg of the TAT-FXN fusion protein or placebo at day 14 and day 28. The dots represent median and the horizontal lines represent 25^{th} and 75^{th} percentiles. The dose-dependent increase from baseline in FXN levels in skin is also summarized in Table 1 below.

TABLE 1
Median change from baseline skin FXN levels
Median Change from Baseline Skin FXN (pg/μg)

| Dose | Day 14 | Day 28 |
|---|---|---|
| 25 mg | 2.81 | 2.28 |
| 50 mg | 5.57 | 3.14 |

Median baseline for subjects was 3.7 pg/μg for cohort 1 and 2.1 pg/μg for cohort 2.

After 14 days of daily dosing, a dose-dependent increase in FXN levels from baseline is observed in skin cells. At 28 days, which is after subjects were switched to every-other-day dosing for 14 days, a dose dependent increase in frataxin levels is still observed, but the magnitude of the increase is lower.

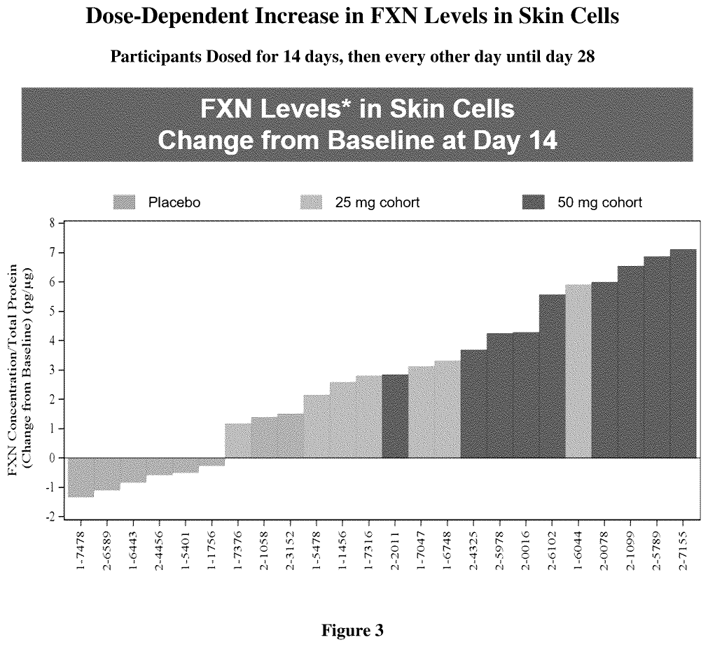

FIG. 3 is a waterfall plot showing the individual changes in FXN levels from baseline in skin cells of subjects with FRDA who were administered 25 mg or 50 mg of the TAT-FXN fusion protein or placebo at day 14. Each bar represents an individual subject. FIG. 3 indicates that the majority of subjects treated with the TAT-FXN fusion protein had significant increases in FXN levels while subjects who were administered placebo had FXN levels that were basically unchanged. There is also a dose response observed, as FXN levels are increased in most of the cohort 2 (receiving 50 mg of TAT-FXN fusion protein), as compared to cohort 1 (receiving 25 mg dose of TAT-FXN fusion protein).

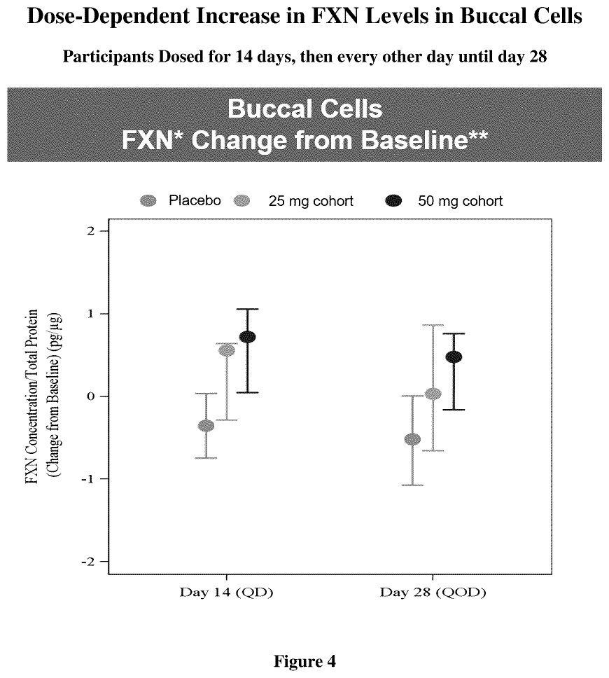

FIG. 4 is a graph showing dose-dependent increase from baseline in FXN levels in buccal cells of subjects with FRDA who were administered 25 mg or 50 mg of the TAT-FXN fusion protein or placebo at day 14 and day 28. The dots represent median and the horizontal lines represent 25^{th} and 75^{th} percentiles. Median baseline for subjects was 1.78 pg/μg for cohort 1 and 1.61 pg/μg for cohort 2.

After 14 days of dosing, a dose-dependent increase in FXN levels from baseline is observed in skin cells, although magnitude of the increase is lower than that observed in skin cells because buccal cells have lower levels of FXN than skin cells. The buccal cells also do not maintain the increased FXN levels following the switch to every other day dosing between day 14 and day 28 for cohort 1. This effect may be due to the quick turnover of buccal cells.

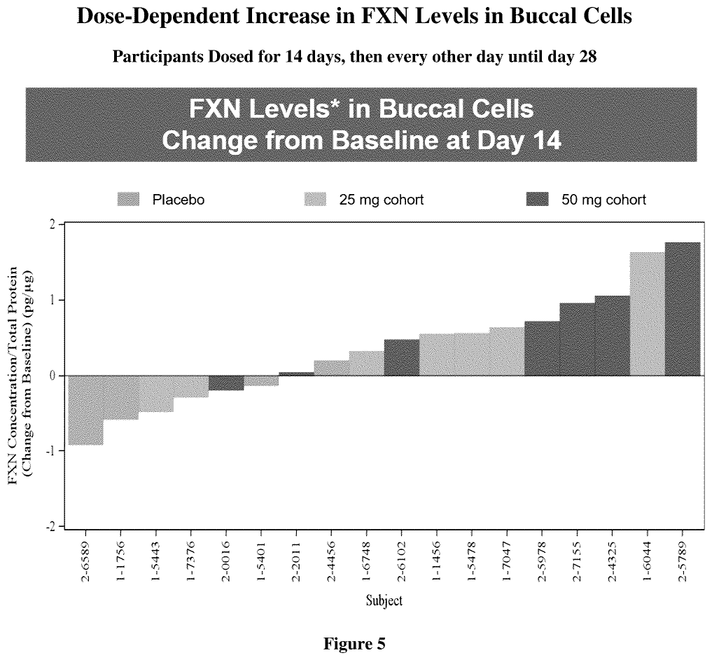

FIG. 5 is a waterfall plot showing the individual changes in FXN levels from baseline in buccal cells of subjects with FRDA that were administered 25 mg or 50 mg of the TAT-FXN fusion protein or placebo at day 14. FIG. 5 indicates that most treated subjects had increases in their FXN levels, while the FXN levels of subjects receiving placebo remained unchanged.

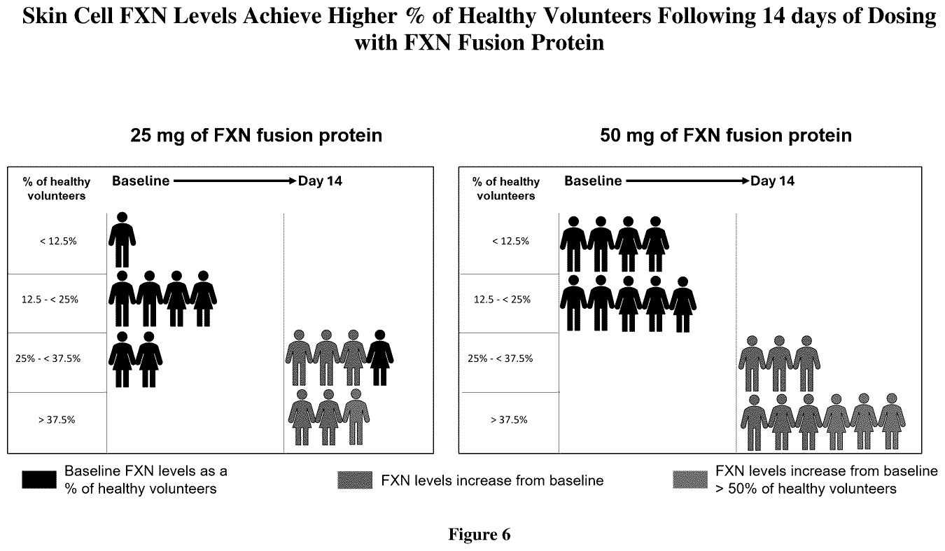

FIG. 6 is a schematic showing the number of subjects who had a shift in their FXN levels in skin cells as a percentage of healthy volunteers after 14 days of treatment. Percentage of healthy volunteers was calculated by dividing each participant's FXN level by the average FXN level from the non-interventional healthy volunteer study (N=60). FIG. 6 includes only those subjects who had quantifiable levels of FXN at baseline and day 14.

The data presented in FIG. 6 indicates that of the 7 subjects who received a daily 25 mg dose of the TAT-FXN fusion protein for 14 days, one subject had a baseline level of FXN in skin cells, and 6 subjects had an increased from their baseline level of FXN in skin cells at day 14. Of the 6 subjects who had an increased from baseline level of FXN at day 14, one subject had an FXN level that is >50% of the FXN level in healthy volunteers.

The data presented in FIG. 6 also indicates that of the 9 subjects who received a daily 50 mg dose of the TAT-FXN fusion protein for 14 days, all 9 subjects had an increased from baseline level of FXN in skin cells at day 14, with 3 subjects having an FXN level that was >50% of the FXN level in healthy volunteers.

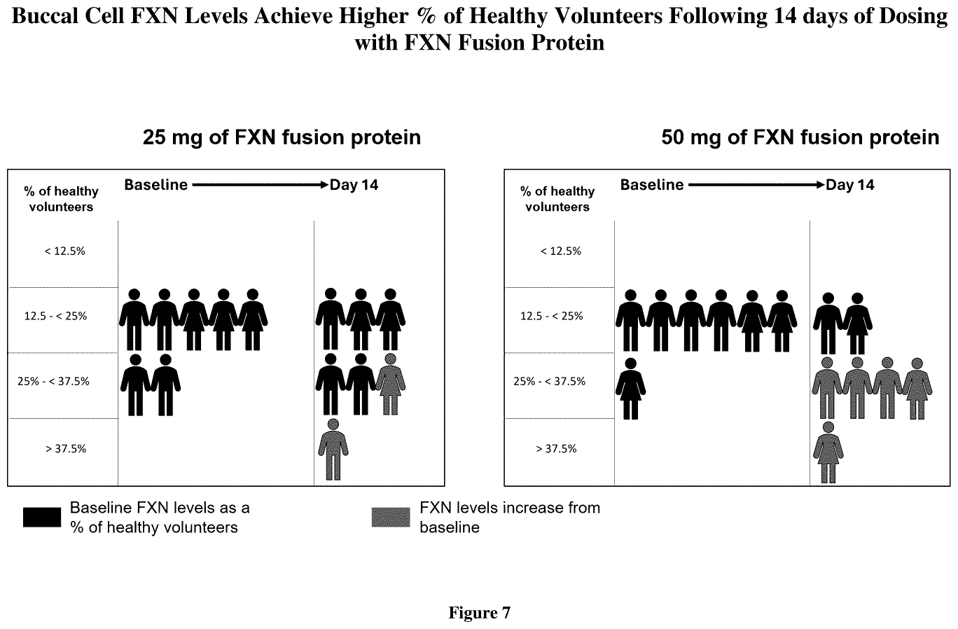

FIG. 7 is a schematic showing the number of subjects who had a shift in their FXN levels in buccal cells as a percentage of healthy volunteers after 14 days of treatment.

The data presented in FIG. 7 indicates that of the 7 subjects who received a daily 25 mg dose of the TAT-FXN fusion protein for 14 days, 5 subjects had a baseline level of FXN in buccal cells, and 2 subjects had an increased from baseline level of FXN in buccal cells at day 14. Of the 2 subjects who had an increased from baseline level of FXN at day 14, one subject had an FXN level that was >37.5% of the FXN level in healthy volunteers.

The data presented in FIG. 7 also indicates that of the 7 subjects who received a daily 50 mg dose of the TAT-FXN fusion protein for 14 days, 2 subjects had a baseline level of FXN in buccal cells and 5 subjects had an increased from baseline level of FXN in buccal cells at day 14. Of the 5 subjects who had an increased from baseline level of FXN at day 14, one subject had an FXN level that was >37.5% of the FXN level in healthy volunteers.

The results presented in Example 1 indicate that once daily administration of the TAT-FXN fusion protein at the dose of 25 mg and 50 mg leads to increased levels of FXN in both skin and buccal samples in subjects with FRDA.

### Example 2. Prediction of Tissue FXN Levels with Long Term Administration of TAT-FXN Fusion Protein in Adults with FRDA Using Modeling and Simulations

### Abstract

### Methods

Plasma concentrations of the TAT-FXN fusion protein and skin FXN concentrations from adults with FRDA before and after short term (<30 days) subcutaneous administration of 25 mg, 50 mg, 75 mg, and 100 mg of an exemplary FXN fusion protein in Phase 1 and 2 clinical studies were used to construct an exposure-response model. Simulations of a population of virtual FRDA patients receiving daily doses of 25, 50, 75 or 100 mg of FXN fusion protein were performed (n=100, 100 trials) and skin FXN profiles over time at each dose were predicted.

### Results

The simulations predicted that skin FXN concentrations should reach steady state at approximately 28 days after daily administrations across all doses. Daily administration of 25 mg, 50 mg, 75 mg, and 100 mg of the TAT-FXN fusion protein was predicted to attain a median maximum skin FXN concentration of 6.22 pg/μg, 9.06 pg/μg, 11.9 pg/μg, and 14.7 pg/μg, respectively.

### Discussion

In a separate study, the mean skin FXN concentration in healthy controls with 2 normal frataxin alleles was 16.35 pg/μg. Prior published studies indicate that the mean FXN concentration in asymptomatic heterozygous carriers is 50% of healthy controls. In relation to these findings, 59% of patients with FRDA receiving daily 50 mg of FXN fusion protein are predicted to achieve skin frataxin concentrations that are equal or above 50% of the concentrations found in healthy controls.

### Conclusion

Modeling and simulation using data from short term studies of FXN fusion protein administration can be used to predict a potential long term therapeutic dose. Daily administration of 50 mg of FXN fusion protein is predicted to result in skin FXN concentrations that are >50% of FXN concentrations found in healthy controls.

### Introduction

FXN deficiency is the root cause of Friedreich's ataxia (FRDA). Mean tissue FXN concentrations in patients with FRDA range from 21% to 35% relative to healthy controls with 2 normal FXN gene alleles. In asymptomatic heterozygous carriers, mean tissue FXN concentrations are approximately 50% of controls. Increases in tissue FXN concentrations were observed in adults with FRDA who received the TAT-FXN fusion protein in short term Phase 1 and phase 2 studies. Over a dose range from 25 mg to 100 mg of the TAT-FXN fusion protein, observed increases in tissue FXN concentrations appeared to be proportional to both dose and plasma concentrations of the FXN fusion protein. The objective of this study was to predict the potential increase in tissue FXN concentrations after long term administration of 25 mg to 100 mg TAT-FXN fusion protein.

### Methods: Clinical Studies

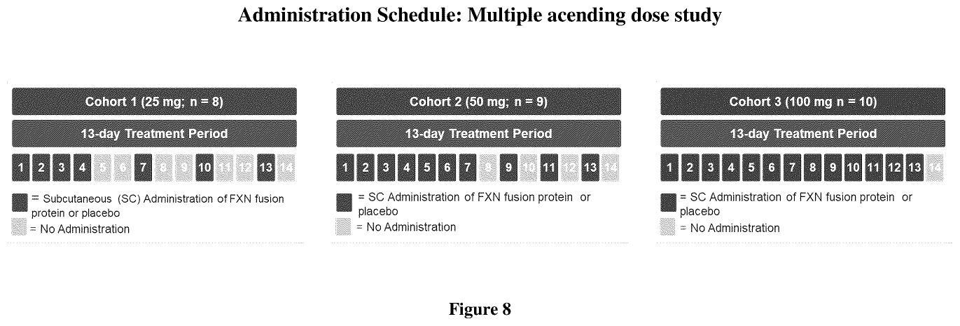

Plasma TAT-FXN fusion protein pharmacokinetic (PK) and skin FXN concentration (pharmacodynamic [PD]) data were collected from 3 completed clinical studies in adults with FRDA. The first study was a single ascending dose study which included dosing with 25 mg, 50, mg, 75 mg, and 100 mg of the TAT-FXN fusion protein and evaluation of the TAT-FXN fusion protein concentrations. The second study was a multiple ascending dose study which included dosing with 25 mg, 50, mg, 75 mg, and 100 mg of the TAT-FXN fusion protein in accordance with the dosing schedule shown in FIG. 8. In the multiple ascending dose study, plasma samples to evaluate concentrations of the TAT-FXN fusion protein were obtained on days 1, 7 and 13, and skin samples to evaluate FXN concentrations were obtained at baseline and on day 13. The third study, which is described in Example 1, was a dose exploration study evaluating 25 mg and 50 mg doses of the TAT-FXN fusion protein administered according to the dosing schedule illustrated in FIG. 1. In this study, full plasma samples were obtained on days 1, 7, 14 and 28 to evaluate TAT-FXN fusion protein PK profiles, and skin samples to evaluate FXN concentration were obtained at baseline and on days 14 and 28.

### Methods: PK/PD

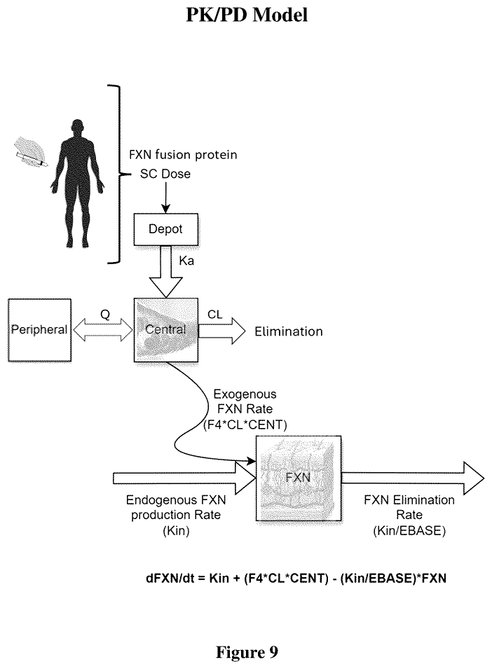

For pharmacokinetic/pharmacodynamics (PK/PD) modeling studies, a populational PK model (popPK) was developed from 1502 PK samples using NONMEM®. Beal's M3 method was applied to allow inclusion of BLQ data. A PK/PD model was developed using 95 non-BLQ post-dose observations collected from 61 unique subjects with FRDA, as shown in FIG. 9. Here, the model parameters applied were Kin (zero order rate constant for FXN) 0.0241 1/h; EBASE (estimated baseline) 3.28 pg/μgₚᵣₒₜ; F4 (proportionality constant for FXN input from central compartment) 0.0194 103 pg/μg/μgₚᵣₒₜ. A population of virtual FRDA patients receiving subcutaneous daily doses of 25, 50, 75 or 100 mg of the TAT-FXN fusion protein for 40 days was simulated (n=100, 100 trials). Body weight and age of diagnosis were simulated based on the observed covariate distributions. Distribution of increases in FXN concentration from 50 sets of 2500 virtual FRDA patients was stratified by baseline FXN concentrations and the age of onset. Model verification was conducted by visual predictive checks (VPCs).

### Results

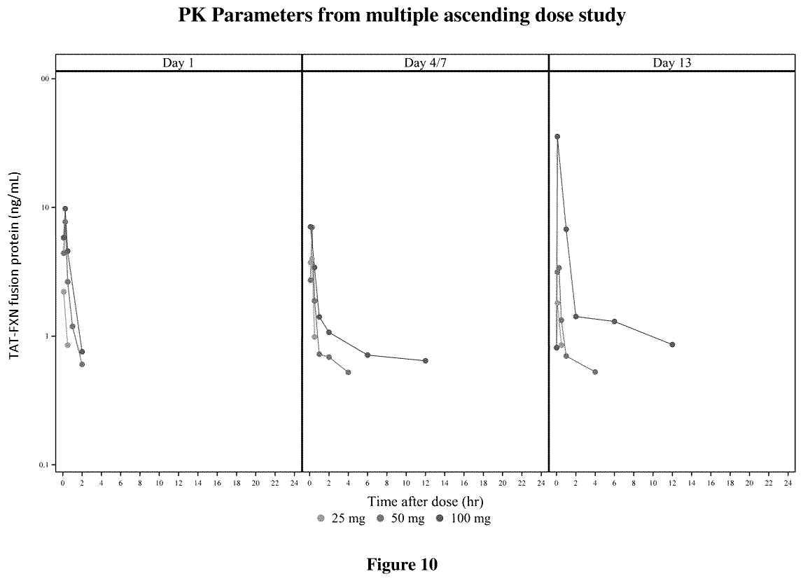

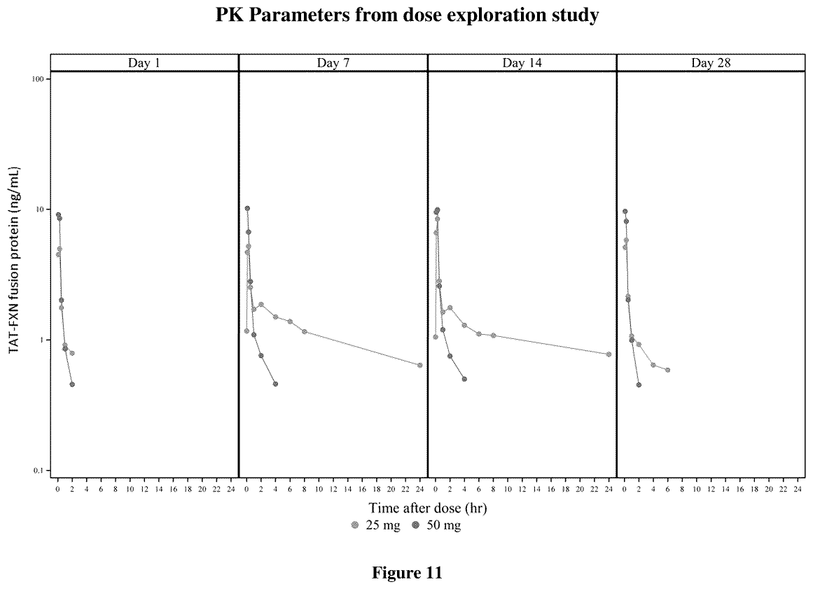

FIG. 10 is a plot showing mean and SD PK parameters with doses of 25 mg, 50 mg and 100 mg for the last day of daily dose for the multiple ascending dose study, while FIG. 11 is a plot showing mean and SD PK parameters with doses of 25 mg and 50 mg for the last day of daily dose for the dose exploration study. The data shown in FIGS. 10 and 11 demonstrates that after subcutaneous administration of the TAT-FXN fusion protein, rapid absorption with a multi-exponential decline in concentrations was observed with dose-proportional increases in exposure.

As illustrated in FIG. 2 and Table 1 of Example 1 for the dose exploration study, and in Table 2 below for the multiple ascending dose study, dose-dependent increases in FXN concentrations were observed in the studies.

TABLE 2
Median change from baseline skin FXN levels in multiple ascending
dose study
Median Change from Baseline Skin FXN (pg/mg)

| Dose | Day 13 |
|---|---|
| 25 mg | 0.867 |
| 50 mg | 2.82 |
| 100 mg | 10.6 |

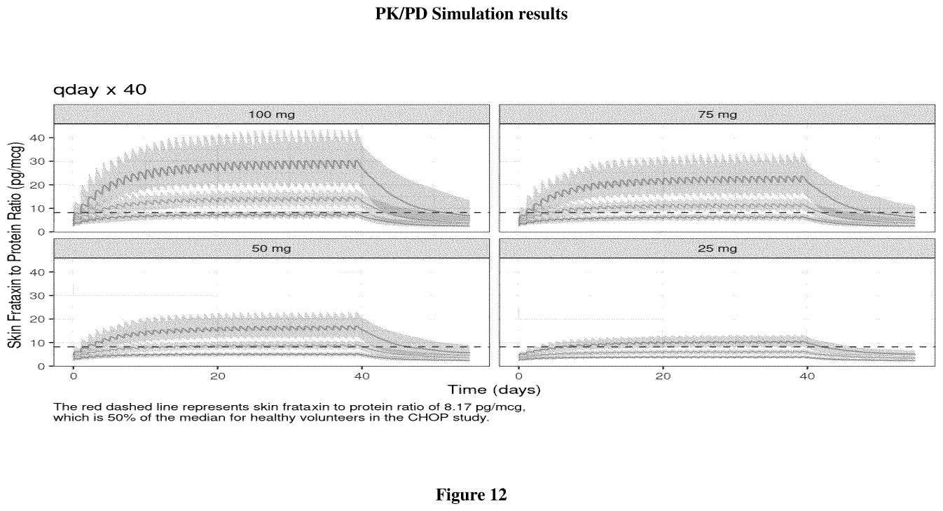

FIG. 12 is a plot showing skin frataxin to protein ratio over time predicted from PK/PD simulations for a dose of 100 mg, 75 mg, 50 mg and 25 mg of TAT-FXN fusion protein. Dashed red line represents 50% of the average skin frataxin/protein ratio (8.17 pg/μg) in a non-interventional study in healthy controls. Blue line represents the median of simulated values across trials, and the red lines are the 10th and 90th percentiles. Shaded regions represent the 95% confidence intervals of the corresponding percentiles (10th, 50th, and 90th).

Median simulated values show in FIG. 12 indicate that 50 mg of the TAT-FXN fusion protein administered daily is predicted to lead to a median increase in FXN concentrations of 5.64 pg/μg from baseline and increase skin FXN concentrations in 59% of simulated patients with FRDA to levels approximating or exceeding values representing 50% of the average skin FXN concentration in healthy controls (red dashed horizontal line).

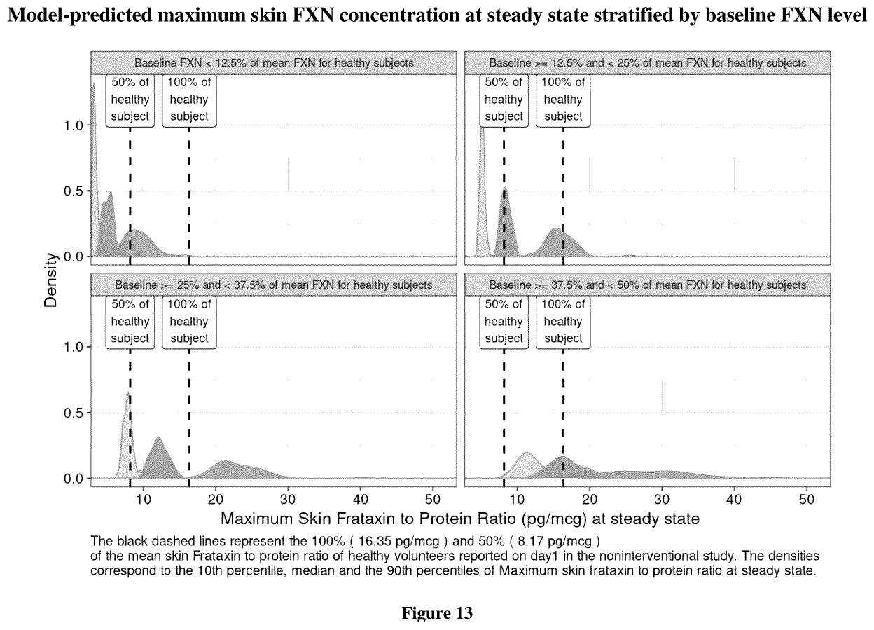

FIG. 13 is a plot showing model-predicted maximum skin FXN concentration at steady state stratified by baseline FXN level. The black dashed lines represent 50% (8.17 pg/mcg) and 100% (16.35 pg/mcg) of the mean skin FXN concentrations in healthy controls. The densities correspond to the 10th percentile (yellow), medians (green), and 90th percentile (red) of the simulated maximum skin FXN to protein ratio at steady state.

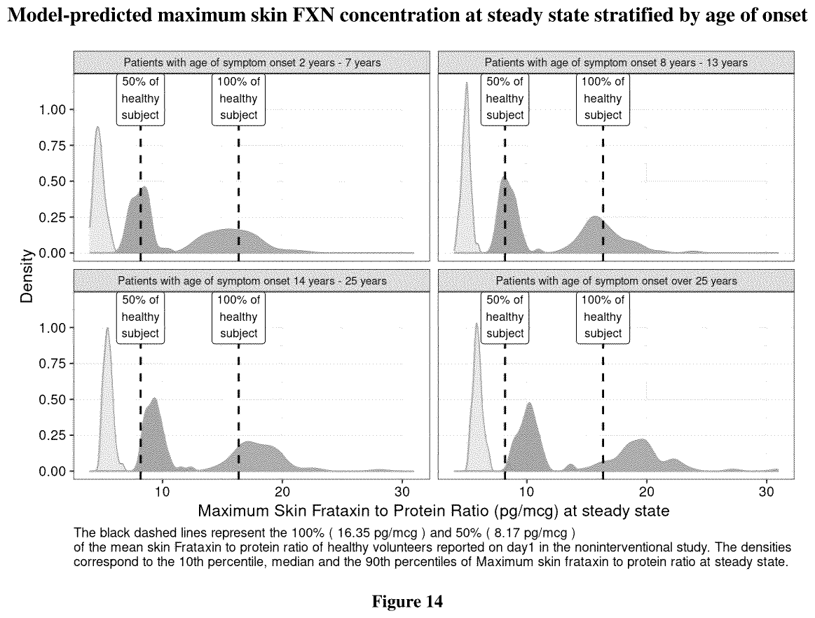

FIG. 14 is a plot showing model-predicted maximum skin FXN concentration at steady state stratified by age of onset. The black dashed lines represent 50% (8.17 pg/mcg) and 100% (16.35 pg/mcg) of the mean skin FXN concentrations in healthy controls. The densities correspond to the 10th percentile (yellow), medians (green), and 90th percentile (red) of the simulated maximum skin FXN to protein ratio at steady state.

Median simulated values shown in FIGS. 13 and 14 indicate that 50 mg of the TAT-FXN fusion protein administered daily is predicted to increase tissue FXN concentrations to a range that approaches or surpasses the 50% level relative to the mean healthy control FXN concentrations in the majority of patients with FRDA regardless of baseline FXN concentration and age of onset.

### Conclusions

In this study, dose-dependent increases in TAT-FXN fusion protein exposure and skin FXN concentrations were observed in adults with FRDA after short term administration of 25 mg to 100 mg of the TAT-FXN fusion protein. Modeling and simulation predicted that daily administration of 50 mg of the TAT-FXN fusion protein would result in skin FXN concentrations that are >50% of concentrations found in healthy controls. Further extension of the model applications to the pediatric population is planned to predict the impact of administration of the TAT-FXN fusion protein on FXN concentrations across different age groups. Data from an ongoing open label extension study will be used to confirm TAT-FXN fusion protein exposure and changes in tissue FXN concentrations after long term administration of TAT-FXN fusion protein to patients with FRDA.

### Example 3. Disease Characteristics and Tissue FXN Concentrations in Adults with FRDA Participating in Interventional Studies of an Exemplary FXN Fusion Protein

### Abstract

### Methods

Disease characteristics, e.g., age at onset and guanine adenine adenine (GAA) repeat length, of adults with FRDA participating in Phase 1 and 2 interventional studies of TAT-FXN fusion protein were summarized and evaluated relative to baseline buccal and skin cell FXN concentrations.

### Results

Sixty-one subjects participated in at least one study; 18 subjects participated in more than one study. Mean age was 31.9 years, and the age range was 19-69 years. Mean age of onset was 15.9 years, and the range was 5-60 years. Mean (range) shorter and longer GAA repeat lengths were 555.8 (99-1000) and 890.2 (265-1300). Mean baseline modified Friedreich's Ataxia Rating scale neurologic score was 49.5 (13.2-74.5). Mean (range) baseline buccal and skin cell frataxin concentrations were 1.90 (0.70-4.95) and 3.25 (1.40-8.10) pcg/μg, respectively. There is a relationship between FXN concentrations and age of onset and GAA repeat length. There is also a relationship between skin and buccal cell FXN concentrations.

### DISCUSSION

Age of onset is associated with more rapid disease progression. Data from the interventional studies of FXN fusion protein are consistent with previously published data suggesting that lower tissue FXN concentrations are associated with more rapid disease progression. Increasing FXN concentrations in patients with FRDA may decrease rate of disease progression.

### Conclusion

The study population in the interventional studies of TAT-FXN fusion protein is representative of the FRDA population, and tissue FXN concentration data from these studies are consistent with prior studies demonstrating that lower FXN concentrations are associated with earlier onset of disease. Buccal and skin cell FXN levels correlate with each other.

### Introduction

Friedreich ataxia (FRDA) is an autosomal recessive neurodegenerative disorder caused by a guanine adenine adenine (GAA) repeat expansion in the FXN gene that results in frataxin (FXN) deficiency. Affected individuals develop a progressive ataxia that leads to a loss of ambulation. Tissue FXN concentrations are correlated with age of disease onset and inversely correlated with the number of GAA repeats and rate of disease progression. The range of the number of GAA repeats observed in the population of patients with FRDA results in a diverse spectrum of disease characteristics.

The goal of this study was to describe the characteristics, including tissue FXN concentrations, in adults with FRDA who participated in Phase 1 and Phase 2 clinical studies evaluating a TAT-FXN fusion protein as a potential treatment for patients with FRDA.

### Methods

Demographics and disease characteristics, as well as skin FXN concentration data, were collected from 3 completed clinical studies in adults with FRDA that are described in Example 2: The first study was a single ascending dose study which included dosing with 25 mg, 50, mg, 75 mg, and 100 mg of the TAT-FXN fusion protein and evaluation of the TAT-FXN fusion protein concentrations. The second study was a multiple ascending dose study which included dosing with 25 mg, 50, mg, 75 mg, and 100 mg of the TAT-FXN fusion protein in accordance with the dosing schedule shown in FIG. 8. In the multiple ascending dose study, plasma samples to evaluate concentrations of the TAT-FXN fusion protein were obtained on days 1, 7 and 13, and skin samples to evaluate FXN concentrations were obtained at baseline and on day 13. The third study, which is described in Example 1, was a dose exploration study evaluating 25 mg and 50 mg doses of the TAT-FXN fusion protein administered according to the dosing schedule illustrated in FIG. 1.

For statistical analyses, a linear regression model was used to investigate the relation of FXN levels in buccal cells to disease severity and progression; the relation of age of onset of disease manifestations to GAA repeat length on the shorter allele; and the relation of frataxin levels in buccal cells to frataxin levels in skin cells.

The demographics and baseline disease characteristics data for adults with FRDA participating in clinical studies of the TAT-FXN fusion protein are summarized in Table 3.

TABLE 3
Summary of demographic and baseline disease characteristics
of adults with FRDA participating in clinical studies

| | N* | Median | Mean | Min | Max |
|---|---|---|---|---|---|
| Age | 61 | 28.0 | 31.9 | 19 | 69 |
| Age of Onset | 61 | 15.0 | 15.9 | 5 | 60 |
| Age of Diagnosis | 61 | 19.0 | 21.0 | 5 | 64 |
| Shorter GAA (GAA₁) | 60 | 550.0 | 555.8 | 99 | 1000 |
| Longer GAA (GAA₂) | 60 | 900.0 | 890.2 | 265 | 1300 |
| Frataxin, % of Control** | 57 | 24.4 | 23.9 | 8.7 | 61.9 |
| mFARS Score | 61 | 52.0 | 49.5 | 13.2 | 74.5 |
| Upright Stability Score | 61 | 32.0 | 26.9 | 7.0 | 35.0 |
| Dominant hand 9-hole peg test | 61 | 71.0 | 84.8 | 26.0 | 229.2 |
| T25-FW Test Score | 51 | 9.9 | 13.4 | 4.3 | 48.5 |
| Left Ventricular Mass (g) | 61 | 163.4 | 168.0 | 73.7 | 398.8 |
| LVEF % | 61 | 63.0 | 63.5 | 52 | 76 |
| Ambulatory Status*** | | | | | |
| No | 36 | | | | |
| Yes | 25 | | | | |

*18 subjects participated in more than 1 study.
**Quantifiable buccal cells FXN levels relative to the median of healthy controls
**Ambulatory status is based on the gait score (E7 = 5 vs. <5) of the upright stability subscore of the mFARS
GAA: guanine adenine adenine;
mFARS: modified Freidreich's ataxia rating scale;
T25FW: timed 25-foot walk;
LVEF: left ventricular ejection fraction

Data on the quantifiable buccal cell FXN concentrations in adults with FRDA who participated in the clinical studies were organized into quartiles as shown in Table 4.

TABLE 4
Summary of disease characteristics of participants in clinical studies

| | FXN | Age at | | | | |
|---|---|---|---|---|---|---|
| | Concentration* | Symptom | Age at | | | Baseline |
| Quartile | (ng/mcg) | Onset** | Diagnosis** | GAA₁** | GAA₂** | mFARS** |
| Q1 | <1.31 | 10.5 | 14.5 | 616.5 | 899.5 | 66.3 |
| (N = 14) | | | | | | |
| Q2 | 1.31-<1.95 | 13.5 | 23.0 | 486.0 | 866.0 | 46.8 |
| (N = 14) | | | | | | |
| Q3 | 1.95-<2.30 | 16.0 | 19.0 | 555.0 | 871.5 | 50.8 |
| (N = 14) | | | | | | |
| Q4 | >=2.30 | 19.0 | 27.0 | 400.0 | 933.0 | 34.5 |
| (N = 15) | | | | | | |

*Quantifiable buccal cells frataxin levels
**Median values
Median buccal cell FXN concentration in healthy controls = 8.1 ng/mcg

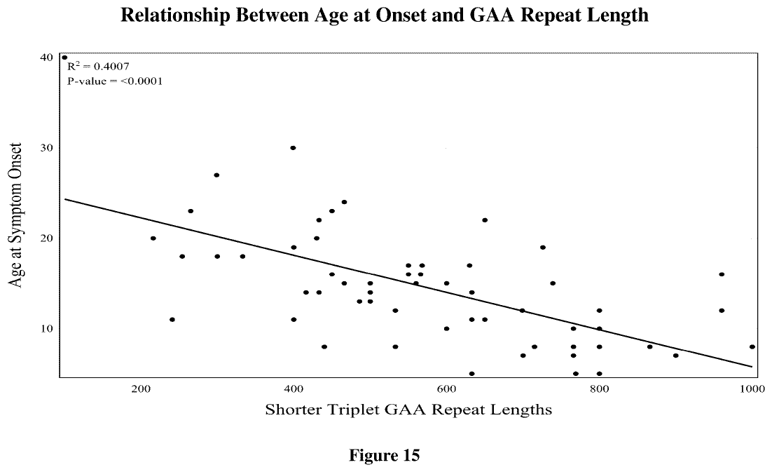

FIG. 15 is a graph showing the relationship between age at onset and shorter triplet GAA repeat length in adults with FRDA participating in the clinical studies with the TAT-FXN fusion protein. The data shown in FIG. 15 indicate that age at onset is inversely correlated with GAA1 repeat length.

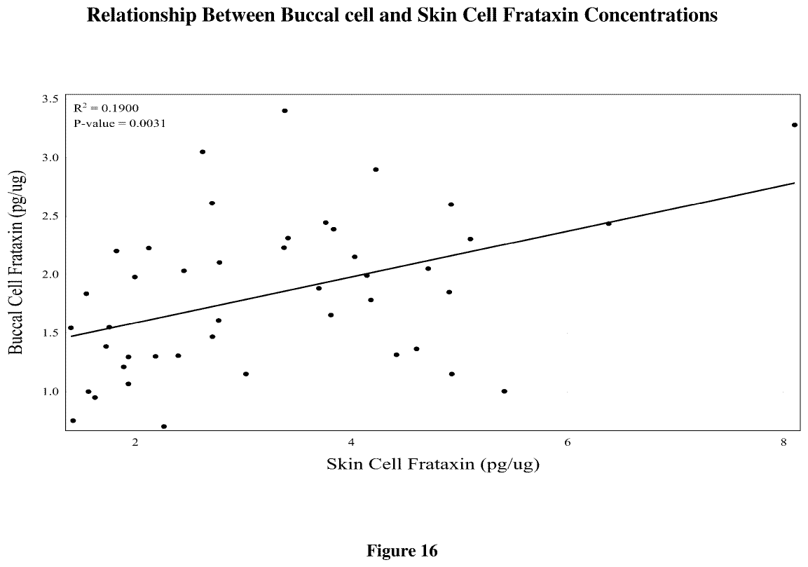

FIG. 16 is a graph showing the relationship between buccal cell and skin cell frataxin concentrations in adults with FRDA participating in clinical studies with the TAT-FXN fusion protein. The data shown in FIG. 16 indicate that buccal cell FXN concentrations correlate with skin cell frataxin concentrations.

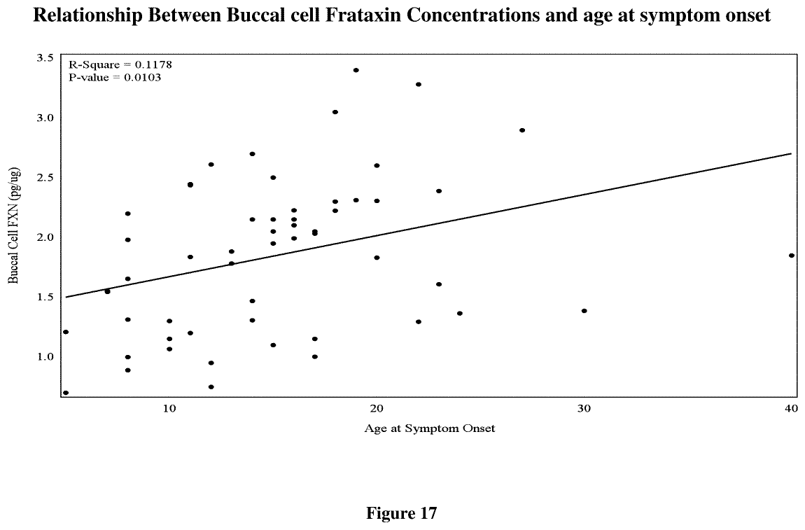

FIG. 17 is a graph showing the relationship between buccal cell frataxin concentrations and age at symptom onset in adults with FRDA participating in clinical studies with the TAT-FXN fusion protein.

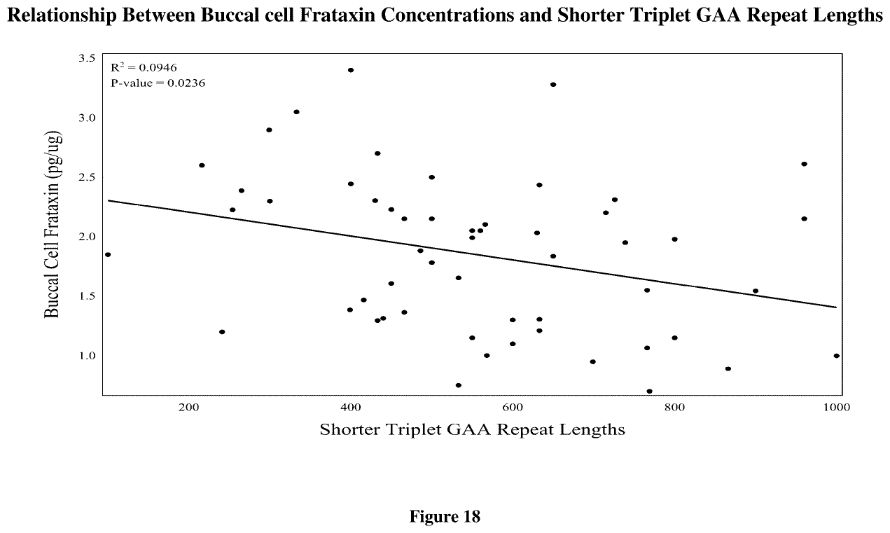

FIG. 18 is a graph showing the relationship between buccal cell frataxin concentration and shorter triplet GAA (guanine adenine adenine) repeat lengths in adults with FRDA participating in clinical studies with the TAT-FXN fusion protein.

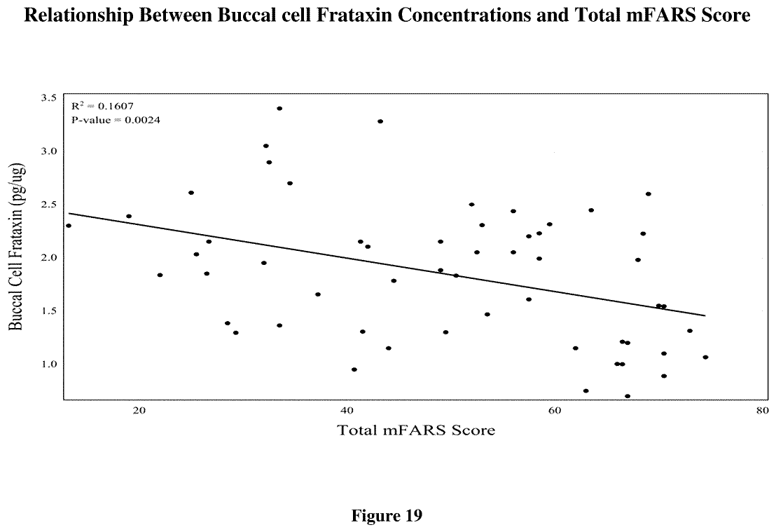

FIG. 19 is a graph showing the relationship between buccal cell frataxin concentration and total modified Freidreich's ataxia rating scale (mFARS) score in adults with FRDA participating in clinical studies with the TAT-FXN fusion protein.

The data shown in FIGS. 17-19 indicate that buccal cell FXN concentrations correlate with age at onset, and inversely correlate with GAA1 repeat length and mFARS score in adults with FRDA participating in clinical studies of the TAT-FXN fusion protein.

### Conclusions

Population of 61 participants in the clinical studies of the TAT-FXN fusion protein represent a wide range of adults with FRDA. Relationships observed between disease characteristics and buccal cell frataxin concentrations in population of participants in the clinical studies are consistent with reports from previous studies. As reported previously, lower tissue frataxin concentrations are associated with more severe disease. Buccal cell FXN concentrations are correlated with skin cell FXN concentrations.

### Example 4. An Open-Label Extension Study to Assess the Long-Term Safety, Efficacy, Pharmacodynamics, Pharmacokinetics, and Tolerability of Subcutaneous TAT-FXN Fusion Protein in Subjects with Friedreich's Ataxia

### Study Protocol

### Background Information

Friedreich's ataxia (FRDA) is the most common inherited ataxia in humans and results from a deficiency of the mitochondrial protein, frataxin (FXN). FRDA is a rare disease with an incidence that is estimated to be 1:29,000, and a carrier frequency of ˜1:85. In the United States (US), there are an estimated 4,000 to 5,000 cases, primarily in Caucasians of European descent, with an equal frequency in males and females. It is a progressive multisystem disease, typically beginning in mid-childhood. Patients suffer from multiple symptoms, including progressive neurologic and cardiac dysfunction. A key feature of FRDA is the primary neurodegeneration of the dorsal root ganglia and the dentate nucleus of the cerebellum leading to the hallmark clinical findings of progressive limb ataxia and dysarthria. A hypertrophic cardiomyopathy is common and associated with early mortality in the third to fifth decade of life. Other clinical findings can include scoliosis, fatigue, diabetes, visual impairment, and hearing loss.

Inheritance is autosomal recessive and is predominantly caused by an inherited guanine adenine adenine (GAA) triplet expansion in the first intron of both alleles of the FXN gene. This triplet expansion causes transcriptional repression of the FXN gene, so patients produce only small quantities of FXN. There is some correlation between GAA repeat number and the onset and severity of clinical symptoms, with higher repeat numbers associated with more severe disease. Heterozygotes (carriers) typically have FXN levels at ˜50% of normal but are phenotypically normal. The FXN levels in whole blood in both heterozygotes and in FRDA patients have been shown to be stable over time.

The TAT-FXN fusion protein of the disclosure (CTI-1601) is being developed as a novel, specific treatment to supplement FXN levels in adults and children with FRDA. Currently, there is no approved FXN replacement therapy available for the treatment of FRDA. The only treatment for FRDA approved by the US Food and Drug Administration (FDA) is omaveloxolone. The precise mechanism by which omaveloxolone exerts its therapeutic effect in patients with FRDA is unknown but has been shown to activate the Nuclear factor (erythroid-derived 2)-like 2 (Nrf2) pathway which is involved in the cellular response to oxidative stress. Omaveloxolone does not appear to affect tissue FXN concentrations, and therefore does not address the core pathophysiology of FRDA.\

### Tat-FXN Fusion Protein—Clinical Information

CTI-1601, intended to be a mitochondrial FXN replacement therapy, is a recombinant fusion protein and is intended to deliver FXN, the protein deficient in FRDA, to the mitochondria where it is intended to increase mitochondrial levels of FXN and restore normal mitochondrial function in patients with FRDA.

CTI-1601 administered SC in a single dose was well tolerated at doses of 25, 50, 75, and 100 mg in the single ascending dose (SAD) study. Twenty-eight subjects participated in the SAD study: 6 in Cohort 1, 7 in Cohort 2, 8 in Cohort 3, and 7 in Cohort 4. Most treatment-emergent adverse events (TEAEs) were Common Terminology Criteria for Adverse Events (CTCAE) Grades 1 and 2 in severity. There were no fatal events or serious adverse events (SAEs) reported in this study.

Injection site reactions (ISRs), all Grade 1 in severity, were the most common types of TEAEs, reported in all (100%) subjects treated with CTI-1601; these events were much less frequent (10%) in subjects who received placebo. The majority of TEAEs of ISRs occurred on the day of study drug administration and were considered related to study drug; most of these events resolved on the same day of onset.

The incidence of injection site findings, including discoloration, tenderness, ecchymosis, and scratch marks, was higher (56%) in CTI-1601-treated subjects compared to none (0%) in placebo subjects. The most common finding was tenderness, reported in 39% of all treated subjects, more frequently reported in subjects treated at a dose of 75 mg. Discoloration was only reported in 17% of all treated subject and only reported in subjects treated at 100 mg. Injection site findings were transient and mostly observed at 6 to 12 hours postdose, with discolored areas diminishing in size as of 12 hours postdose and thereafter. Perceived pain at the injection site was minimal and temporary; no subjects perceived pain on Day 2 postdose and thereafter.

The multiple ascending dose (MAD) study consisted of 3 dosing regimen cohorts. Cohort 1 received either placebo or 25 mg administered daily for 4 days followed by a dose administered every 72 hours on Days 7, 10, and 13. Cohort 2 received either placebo or 50 mg administered daily for 7 days followed by a dose administered every 48 hours on Days 9, 11, and 13. Cohort 3 received either placebo or 100 mg administered daily for 13 days. Twenty-seven subjects (11 de novo and 16 who also participated in the SAD study) participated in the MAD study: 8 in Cohort 1, 9 in Cohort 2, and 10 in Cohort 3. One subject in Cohort 2 withdrew from the study on Day 2 due to episodes of nausea and vomiting.

Consistent with the observations from the SAD study, CTI-1601 was safe and well tolerated. Adverse events (AEs) were generally mild, brief, and self-limited, and no SAEs were observed. The most commonly observed AEs were ISRs, which were similar in nature to the ISRs observed in the SAD study and generally resolved. Other AEs that occurred in more than 1 subject included gastrointestinal complaints (nausea, vomiting, diarrhoea) and headache. These AEs occurred sporadically across the 3 cohorts, and the frequency or severity of these AEs did not change with increasing dose. One subject in Cohort 2 (50 mg) discontinued the study after the first dose because of Grade 2 nausea and Grade 1 vomiting which was considered drug related. An increase in buccal and skin cell FXN concentrations was observed in subjects who received 50 mg CTI-1601 daily for 7 days and 100 mg CTI-1601 daily for 13 days.

Prior clinical studies of CTI-1601 have evaluated administration of CTI-1601 daily at doses up to 100 mg for 13 days. In addition, a Phase 2 dose exploration study has evaluated administration of 25 mg and 50 mg CTI-1601 daily for 14 days followed by every other day administration for an additional 14 days. An increase in buccal and skin cell FXN concentrations was observed in subjects who received 25 mg and 50 mg CTI-1601 daily for 14 days. Similar to the SAD and MAD studies, CTI-1601 was safe and well tolerated. Adverse events (AEs) were generally mild, brief, and self-limited, and no SAEs were observed. The most commonly observed AEs were ISRs, which were generally similar in nature to the ISRs observed in the SAD and MAD studies and typically were mild, self-limited, and brief in duration. However, approximately half of subjects experienced pruritis that lasted more than 24 hours at more than 1 injection site. Three subjects in this study experienced a generalized allergic reaction. These reactions included generalized urticaria which required medical treatment. One subject experiencing urticaria received treatment with antihistamines for several days and study drug administration was paused until the urticaria resolved. When study drug was restarted, the subject received an antihistamine prior to study drug administration and again developed urticaria as well as wheezing and shortness of breath requiring supplemental oxygen and treatment with additional antihistamines and steroids. This subject was withdrawn from the study. Another subject developed generalized urticaria which resolved 1 day after treatment with an antihistamine. The subject resumed study drug administration along with pretreatment with an antihistamine and the urticaria did not recur. The third subject developed generalized urticaria relatively late in the treatment period. Despite treatment with antihistamines and corticosteroids the urticaria had not completely resolved until after the end of the treatment period and thus this subject did not resume study drug administration.

The current study will evaluate long-term safety, tolerability, and PK as well as the effect of long-term administration of CTI-1601 on tissue FXN concentrations, pharmacodynamics (PD), and clinical measures of efficacy in patients with FRDA. This study will provide CTI-1601 to subjects who participated in a prior CTI-1601 study as part of the clinical development program.

### Rationale for the Study

Patients with FRDA have a deficiency of FXN, a protein that is essential for mitochondrial metabolism. CTI-1601, intended to be a mitochondrial FXN replacement therapy, utilizes a cell-penetrant peptide fused to human FXN in order to deliver FXN to the mitochondria where it is presumed to be processed to mature FXN and subsequently become active in mitochondrial metabolism. The specific rationale for this OLE study is to collect long-term safety, tolerability, PK, PD, and efficacy data while allowing subjects who participated in a prior study to continue receiving CTI-1601. This study will also allow those subjects who participated in a prior study and received placebo to initiate treatment with CTI-1601.

### Safety Objectives

To evaluate the safety and tolerability of long-term SC administration of CTI-1601 in subjects with FRDA.

### Efficacy Objectives

To evaluate the effect of long-term SC administration of CTI-1601 on tissue FXN concentrations.
To evaluate the effect of long-term SC administration of CTI-1601 on clinical evaluations of FRDA as assessed by 9-hole peg test (9-HPT), the timed 25-foot walk (T25-FW) test, the modified Friedreich's Ataxia Rating Scale (mFARS) and upright stability subscale examinations, and the Friedreich's Ataxia Rating Scale-Activities of Daily Living (FARS-ADL), compared to an external control group.
To evaluate the effect of long-term SC administration of CTI-1601 on other clinical evaluations of FRDA as assessed by the Patient Global Impression of Change (PGI-C), Clinical Global Impression of Change (CGI-C), Modified Fatigue Impact Scale (MFIS), Functional Staging for Ataxia, left ventricular ejection fraction (LVEF), left ventricular end-diastolic volume (LV EDV), and hemoglobin Alc (HbAlc) over time.

### Pharmacokinetic and Pharmacodynamic Objectives

To evaluate the PK of long-term SC administration of CTI-1601.
To evaluate the effect of long-term SC administration of CTI-1601 on gene expression and selected lipids.

### Study Endpoints

Safety and tolerability of CTI-1601 will be assessed using:
Incidence and severity of TEAEs;
Change from baseline in clinical laboratory data tests (including hematology, biochemistry, HbAlc, lipid profile, and urinalysis);
12-lead electrocardiogram (ECG) monitoring;
Echocardiogram (ECHO) parameters;
Change from baseline in vital signs (VS);
Physical examinations; and
Responses to the Columbia Suicide Severity Rating Scale (C-SSRS).

### Efficacy Endpoints

Efficacy of CTI-1601 will be assessed using:
Change from baseline at each collection timepoint in tissue FXN concentrations normalized to total protein observed in buccal cells collected from cheek swabs, and skin cells collected from skin punch biopsies;
Motor function as assessed by the 9-HPT
Neurologic function as assessed by the mFARS total score;
Motor function as assessed by the T25-FW test (for those who can perform this test);
Neurological function as assessed by the mFARS and upright stability subscale examinations;
ADLs reported by the subject or caregiver as assessed by the FARS-ADL;
Change from baseline in the total fatigue score and all the subscales score as assessed by the MFIS;
Assessment of disease as assessed by the Functional Staging for Ataxia;
Overall impression of change as assessed by the subject using the PGI-C scale;
Overall impression of change as assessed by the clinician using the CGI-C scale;
Cardiac function as assessed by LVEF and LV EDV obtained from an ECHO; and
HbAlc laboratory results.

### Pharmacokinetic Endpoints

Blood samples for population PK analyses will be collected at specified timepoints. Samples will be analyzed for plasma levels of CTI-1601 using a validated bioanalytical method. The date/time will be recorded for each sample as will the date/time of every dose of study drug.

Blood will be drawn at the following timepoints:
Days 1, 30, 60, and 90: predose (within 1 hour prior to dose administration), 5, 15, and 30 minutes, and 1, 2, 4, 6, and 8 hours (serial PK) after the dose.
Day 180: Predose (within 1 hour prior to dose administration) and at 5 and 15 minutes after the dose.
Days 270, 360, & every 3 months (Q3M) thereafter: predose (within 1 hour prior to dose administration).
Early Termination (ET) or study Termination (ST) visit: Study drug administration will not occur at the ET or ST visit. The PK sample should be drawn at roughly the same time predose PK samples were drawn during previous visits. If ET or ST visit occurs later than 30 hours after the last dose administered, the PK sample will not be collected.

The PK endpoints will include but not be limited to the following PK parameters during a dosing interval:
AUC₀₋ₜ: area under the concentration-time curve from time 0 to the time of last quantifiable concentration;
Cₘₐₓ: maximum observed concentration
Tₘₐₓ: time of maximum observed concentration
C_{trough}: concentration reached immediately before the next dose is administered.

### Pharmacodynamic Endpoints

Assessments of potential biomarkers will be performed by measuring changes from prestudy baselines in gene expression and specialized lipids. PD assessments are the following:
Gene expression levels in buccal cells collected from cheek swabs; and
Lipids in plasma derived from whole blood.

### Anti-Drug Antibodies Endpoint

Anti-drug antibodies (ADAs) to CTI-1601 will be measured.

### Study Design

All subjects will receive CTI-1601 in this study. To provide for a surrogate placebo comparator group, an external control group will be constructed from data collected from participants in the Friedreich's Ataxia Clinical Outcome Measures Study (FACOMS), a natural history study for FRDA that collects information from an annual evaluation that includes the mFARS and upright stability subscale examinations, the T25-FW test, and the 9-HPT, if enough data are available from the natural history study.

Subjects who complete a prior study of CTI-1601, regardless of whether the subject took CTI-1601 or placebo, will be eligible to participate in this study unless the subject experienced one or more of the following in a previous CTI-1601 study:
SAE related to study drug;
Significant AE, defined as Grade 3 or higher according to the CTCAE, Version 5.0 or higher, related to study drug;
Some other event, related to participation in a previous study with CTI-1601, that supports the exclusion of the subject from participating in this study as determined by the sponsor (i.e., an AE considered clinically significant by the sponsor regardless of whether it met SAE criteria and regardless of CTCAE grade); and
Withdrawal from participation in a previous study of CTI-1601 for any reason.

A subject will not be screened until all assessments pertaining to the prior study have been completed.

Concomitant use of omaveloxolone (an approved treatment for patients with FRDA) will be permitted in this study. Subjects who are currently receiving omaveloxolone or intend to receive omaveloxolone must either receive CTI-1601 for 6 months prior to their first dose of omaveloxolone or receive omaveloxolone for 6 months prior to their first dose of CTI-1601. Subjects already receiving omaveloxolone must provide documentation of their first pharmacy fulfillment date for omaveloxolone. This date will be used as the start date.

If the omaveloxolone fulfillment date is greater than or equal to 6 months relative to their estimated Day 1 visit for this study, the subjects will proceed with the screening assessments described beginning with Day-42. If the omaveloxolone fulfillment date is less than 6 months relative to their estimated Day 1 visit for this study, the subject will participate in a screening period of up to 6 months. This will include monthly calls from study staff to collect information on AEs and ongoing medications until 2 months prior to initiating treatment with CTI 1601. Subjects will then proceed with the Screening visit described for Day-30. Subjects who are not receiving omaveloxolone at the Screening visit for this study will not be permitted to use omaveloxolone concomitantly until after they have completed 6 months of treatment with CTI-1601. Subjects will proceed with the screening assessments described beginning with Day-42.

Screening assessments will be performed by a combination of on-site and telehealth (TH) visits. On-site Screening assessments (safety laboratory tests, the physical examination, ECG, and ECHO), must be completed with results available within 30 days of Day 1. In addition, a TH visit will be completed within approximately 2 weeks of the Day-1 visit. A freezer and study drug will be sent to the subject prior to the subject traveling to the site for the Day-1 visit. At the Day 1 visit, the study site or a trained subject/caregiver will prepare and administer the first dose of CTI-1601 to eligible subjects. Subjects will receive training to self-administer CTI-1601 SC if the subject has the dexterity and visual acuity to perform SC injections; otherwise, a caregiver committed to preparing and administering the injections will be trained. A back-up caregiver(s) may also be trained. Anatomic areas used as injection sites will be assessed at each scheduled visit and any associated AEs will be recorded as appropriate.

Subject safety will be assessed monthly via remote contact (TH) or OS clinic visits for the duration of the study. Most of the efficacy assessments will be completed Q3M starting at Day 90. Two Follow-up visits (after a subject discontinues study drug entirely will be performed by a visiting nurse 7 days and 30 days post last dose with a window of +3 days. All visits will occur at the study site except for the TH visits (which will occur between on-site visits) and the Follow-up visits completed by the visiting nurse and by study site staff via TH.

Study drug (CTI-1601) will be administered SC QD. The concentration of the CTI-1601 dose formulation for SC injection is 50 mg/mL. The dose planned for this study (25 mg) appears to have been safe and well tolerated in patients with FRDA who participated in previous clinical studies.

### Study Drug

CTI-1601 is a recombinant fusion protein consisting of a cell penetrant peptide linked to the complete FXN protein. CTI-1601 will be provided in liquid form in a 2.0 mL vial with each vial containing 1.25 mL of product at a concentration of 50 mg/mL. CTI-1601 contains the following excipients: histidine, sucrose, polysorbate 20, and sterile water. CTI-1601 will be administered SC QD, via syringe filled by trained subject/caregiver to contain the appropriate amount to be administered.

Study drug (25 mg CTI-1601) administration will occur QD at approximately the same time in the morning on each dosing day.

### Efficacy Assessments

### Friedreich's Ataxia Rating Scale

The FARS comprises a functional disability measure of ataxia, an ADL scale, and a neurological rating scale. This study will only focus on 2 subscores derived from the full FARS questionnaire: mFARS (and its upright stability subcomponent) and FARS-ADL. The mFARS is a modified FARS neurological rating scale involving direct subject participation and targets specific areas impacted by FRDA (bulbar, upper limb, lower limb, and upright stability), with scores ranging from 0 to 93 points, with higher scores indicating a greater level of disability. The mFARS excludes subscale D (peripheral nervous system) and the first 2 questions of subscale A (bulbar) from the neurological rating scale of the FARS questionnaire.

The FARS_ADL score is a FARS rating scale assessing subject ability to complete ADLs (e.g., speech, cutting food, dressing, and personal hygiene), with scores ranging from 0 to 36 points, with higher scores indicating more impairment when completing ADLs. The respondent options are 1) the subject; 2) a combination of the subject and family; or 3) family member or spouse or caregiver for those subjects unable to complete the test. The respondent must be the same throughout the subject's participation in the trial.

### The 9-Hole Peg Test (9-HPT)

The 9-HPT is a quantitative measure of upper extremity (arm and hand) function. Its use with multiple sclerosis (MS) subjects was first reported in 1988, and it has seen increasing use in MS clinical trials as part of the Multiple Sclerosis Functional Composite (MSFC) score as well as in FACOMS and clinical practice. Both the dominant and nondominant hands will be tested twice (2 consecutive trials of the dominant hand, followed immediately by 2 consecutive trials of the nondominant hand). All 4 trials must be performed.

### Timed 25-Foot Walk Test (T25-FW)

The T25-FW test is a quantitative measure of lower extremity function. The T25-FW was incorporated as part of a composite score for MS in 1996 as a recommendation by the National MS Society Clinical Outcomes Assessment Task Force. It has seen increased use in MS clinical trials as part of the MSFC score, as well as in FACOMS and clinical practice.

The T25-FW test will be performed by those who can complete it, with or without assistive devices. Those subjects requiring the use of a self-propelled wheelchair will be excluded from performing the test at the Baseline (Day-1) visit and subsequent visits during the study. It is possible that some subjects may be able to perform the test at the beginning of the study and then subsequently lose the ability to do so. The T25-FW test will be performed twice (once in each direction). The timing component will be measured to a tenth of a second.

### Patient Global Impression of Severity and of Change

The PGI-S is a question that requires the subject to rate the severity of their condition.

The PGI-C is a question depicting a subject's rating of their overall improvement from baseline with study drug.

### Clinical Global Impression of Severity and of Change

The CGI-S is a question that requires the clinician to rate the severity of the subject's condition. The CGI-C is a question intended to measure change in clinical status (symptoms and functional ability) of the subject's condition from Baseline with study drug.

### Functional Staging for Ataxia

Functional Staging for Ataxia will be completed by the PI (or trained subinvestigator). The responses range from normal (Stage 0) to confined to a wheelchair or bed with total dependency for all activities of daily living (Stage 6.0). Increments of 0.5 are permitted.

### Modified Fatigue Impact Scale (MFIS)

The MFIS is a modified form of the Fatigue Impact Scale based on items derived from interviews with MS patients concerning how fatigue impacts their lives. This instrument provides an assessment of the effects of fatigue in terms of physical, cognitive, and psychosocial functioning. The full-length MFIS consists of 21 items. The MFIS is a structured, self-report questionnaire that the patient can generally complete with little or no intervention from an interviewer. However, patients with visual or upper extremity impairments may need to have the MFIS administered as an interview. Interviewers should be trained in basic interviewing skills and in the use of this instrument. The total score for the MFIS is the sum of the scores for the 21 items. Individual subscale scores for physical, cognitive, and psychosocial functioning can also be generated by calculating the sum of specific sets of items.

### Left Ventricular Ejection Fraction and Left Ventricular End-Diastolic Volume

ECHO data will be collected to assess cardiac function for purposes of safety surveillance. Changes in LVEF and LV EDV will also be evaluated over time as an efficacy endpoint.

### Hemoglobin Alc

Hemoglobin Alc will be collected. Changes in HbAlc will be evaluated as a safety endpoint as well as an efficacy endpoint.

### Pharmacokinetic Procedures

Blood samples will be collected to measure plasma concentrations of CTI-1601. Actual PK blood sample collection times versus time of dosing will be monitored and recorded.

Measures will be assessed to quantify tissue FXN concentrations as well as potential biomarkers. Specimen analyses for FXN concentrations and gene expression will be completed using buccal cells obtained from cheek swabs. Skin cells obtained from skin punch biopsy procedures will assess FXN concentrations. Plasma separated from blood will assess specialized lipids.

Two buccal swab systems will be used to collect cells for protein quantitation and gene expression characterization, respectively. Samples will be harvested; 1 sample from the left cheek for protein concentration and 1 sample from the right cheek for gene expression. Samples will be immediately stabilized according to purpose and the swab system manufacturer's directions.

Blood will be used to measure changes in specialized lipids composition. Samples will be prepared and immediately stabilized. Subjects must stop eating and drinking, except for water, 10 hours prior to PD blood lipid collections.

Subjects will undergo skin cell collection procedures. The method of collection will be a skin punch biopsy. A sample 4 mm in diameter and full thickness will be harvested from an area per the investigator's discretion and discussion with the subject. The specimen will be immediately stabilized. The specimen will be used to measure protein concentration.

### Topline Results

At the time of data cut off for this open label extension (OLE study), 14 adults with FRDA were included with up to 260 days of long-term daily treatment with 25 mg of CTI-1601. Among these patients, 57% were nonambulatory, and the subjects were ≥18 years old. The median tissue frataxin level at baseline was 1.13 pg/μg and 2.41 pg/μg in buccal and skin cells, respectfully.

### Safety

Nomlabofsp was generally well tolerated with two serious adverse events which were resolved in 24 hours. Most common adverse events were mild and moderate injection site reactions.

### Frataxin Levels

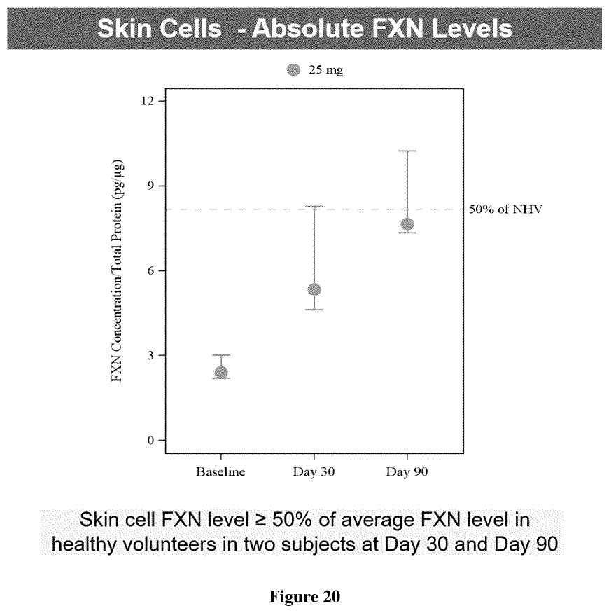

FIG. 20 is a graph showing frataxin levels (pg/μg of total protein) in skin cells measured at baseline, at Day 30 and Day 90 following daily administration of 25 mg of CTI-1601. The dotted line indicates frataxin levels that are 50% of the frataxin levels in healthy volunteers.

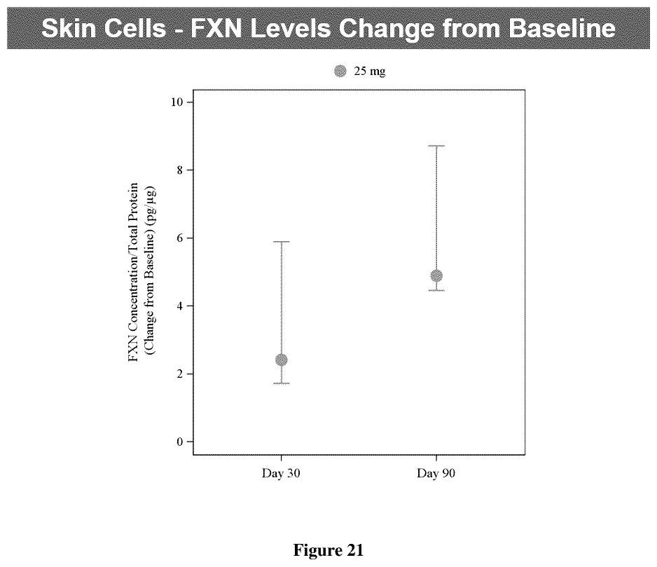

FIG. 21 is a graph showing change from baseline in frataxin levels (pg/μg of total protein) in skin cells measured at Day 30 and Day 90 following daily administration of 25 mg of CTI-1601.

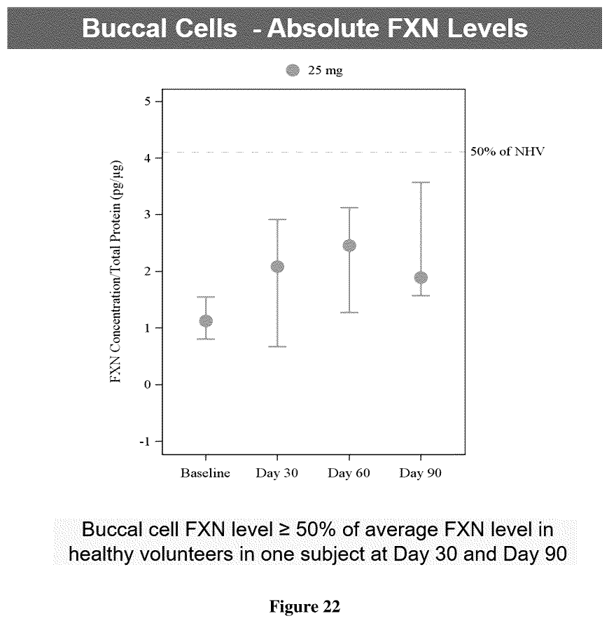

FIG. 22 is a graph showing frataxin levels (pg/μg of total protein) in buccal cells measured at baseline, at Day 30, Day 60 and Day 90 following daily administration of 25 mg of CTI-1601. The dotted line indicates frataxin levels that are 50% of the frataxin levels in healthy volunteers.

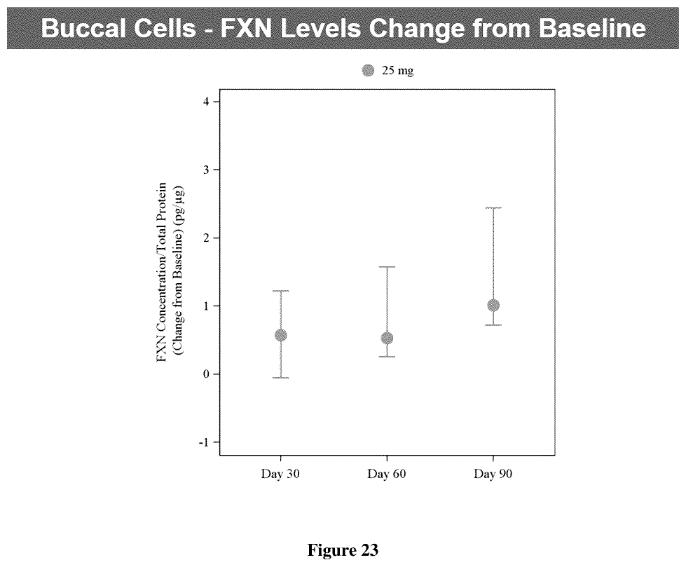

FIG. 23 is a graph showing change from baseline in frataxin levels (pg/μg of total protein) in buccal cells measured at Day 30, Day 60 and Day 90 following daily administration of 25 mg of CTI-1601.

The median tissue frataxin levels at Day 90 were 1.89 pg/μg in buccal cells and 7.65 pg/μg in skin cells. The median and mean absolute frataxin levels measured in buccal cells and 5 skin cells are also shown in Tables 5-7 below.

TABLE 5
Median frataxin levels in buccal cells and skin cells measured
following daily administration of 25 mg CTI-1601

| | Buccal FXN Levels (pg/μg) | | Skin FXN Levels (pg/μg) | |
|---|---|---|---|---|
| | Median | IQR | Median | IQR |
| Baseline | 1.13 | (0.81, 1.55) | 2.41 | (2.20, 3.01) |
| Day 30 | 2.08 | (0.68, 2.92) | 5.34 | (4.62, 8.28) |
| Change from | 0.58 | (−0.05, 1.22) | 2.42 | (1.73, 5.90) |
| Baseline | | | | |
| Day 60 | 2.46 | (1.28, 3.13) | NC | NC |
| Change from | 0.53 | (0.26, 1.58) | NC | NC |
| Baseline | | | | |
| Day 90 | 1.89 | (1.57, 3.58) | 7.65 | (7.34, 10.25) |
| Change from | 1.01 | (0.72, 2.45) | 4.89 | (4.47, 8.72) |
| Baseline | | | | |

IQR: Interquartile range (P25th, P75th)
NC: Skin biopsies were not collected on Day 60 per the protocol

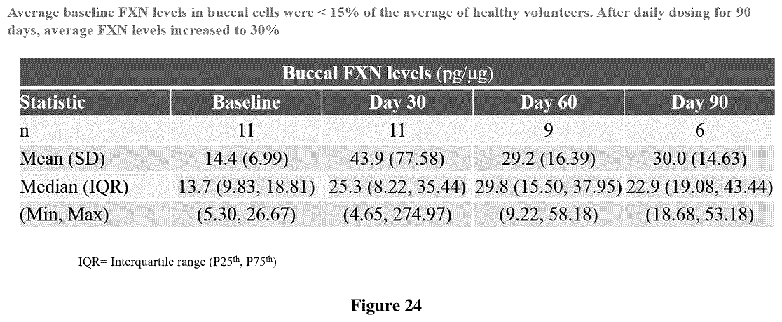

1 (FIG. 24 shows increase in frataxin levels in buccal cells as a percentage of average frataxin levels in healthy volunteers at baseline, Day 30, Day 60 and Day 90 following daily administration of 25 mg of CTI-1601. The average baseline FXN levels in buccal cells were <15% of the average of healthy volunteers. After daily dosing for 90 days, average FXN levels increased to 30%.

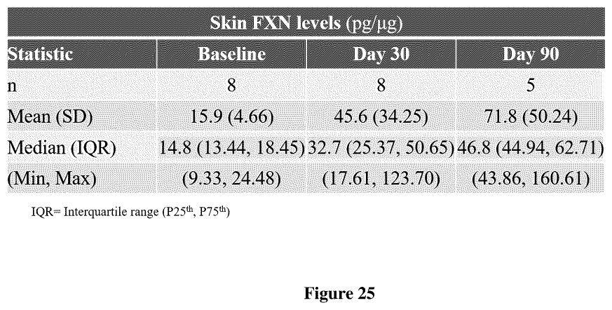

FIG. 25 shows increase in frataxin levels in skin cells as a percentage of average frataxin levels in healthy volunteers at baseline, Day 30 and Day 90 following daily administration of 25 mg of CTI-1601. The average baseline FXN levels in skin cells were <16% of the average of healthy volunteers. After daily dosing for 90 days, average FXN levels increased to 72%.

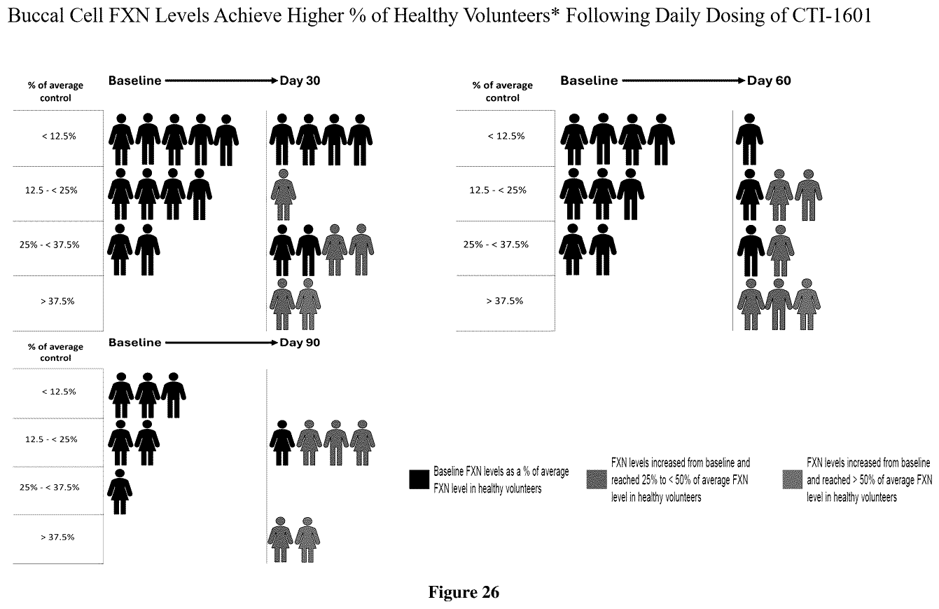

FIG. 26 is a schematic showing the number of subjects who had a shift in their frataxin levels in buccal cells as a percentage of frataxin levels of healthy volunteers following 30 days, 60 days or 90 days of daily dosing with the TAT-FXN fusion protein.

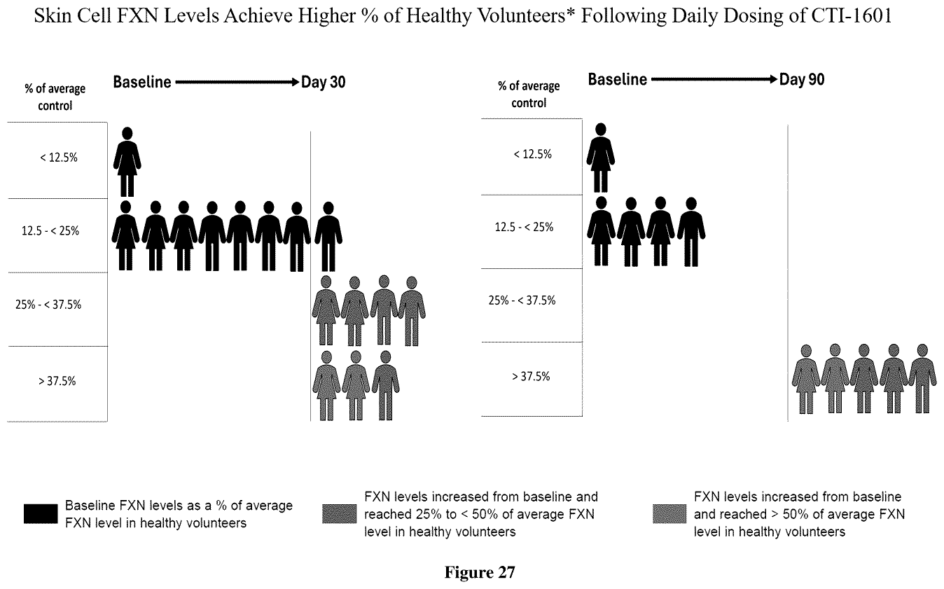

FIG. 27 is a schematic showing the number of subjects who had a shift in their frataxin levels in skin cells as a percentage of frataxin levels of healthy volunteers following 30 days, or 90 days of daily dosing with the TAT-FXN fusion protein.

Daily administration of 25 mg of CTI-1601 increased and maintained tissue frataxin levels over time, reaching 30% and 72% of the frataxin levels in healthy volunteers at day 90 in buccal and skin cells, respectfully. Tissue FXN levels appear to reach steady-state levels by 30 days in buccal cells.

The pharmacokinetic data indicates that there is quick absorption of CTI-1601 after subcutaneous administration. Dosing appeared to reach steady-state by 14 days with no further accumulation.

### Example 5. An Open-Label Extension Study to Assess the Long-Term Safety, Efficacy, Pharmacodynamics, Pharmacokinetics, and Tolerability of Subcutaneous TAT-FXN Fusion Protein in Subjects with Friedreich's Ataxia

### Study Protocol

### TAT-FXN Fusion Protein—Clinical Information

See the section entitled “TAT-FXN Fusion Protein-Clinical Information” for Example 4.

In this ongoing open-label extension (OLE) study (CLIN-1601-201) described in Example 4, 1 subject developed acute anaphylaxis 30 minutes after receiving the first dose of CTI-1601 on Day 1. This subject was withdrawn from the study and did not receive any more doses of CTI-1601. Another subject who had received approximately 44 days of CTI-1601 dosing had a brief seizure less than a minute after receiving a dose of CTI-1601. The seizure ended within 2 minutes, and the subject was admitted to the hospital. No more seizures were observed, and the subject was discharged a few days later. The subject chose to withdraw from the study.

### Rationale for the Study

See the section entitled “Rationale for the Study” for Example 4.

### Study Objectives

### Primary Objective

To evaluate the effect of long-term SC administration of CTI-1601 on tissue FXN concentrations.

### Secondary Objectives

To evaluate the effect of long-term subcutaneous (SC) administration of CTI-1601 on clinical evaluations of FRDA, compared to an external group, as assessed by:
Modified Friedreich's Ataxia Rating Scale (mFARS) and upright stability subscale examinations;
9-hole peg test (9-HPT);
Timed 25-foot walk (T25-FW) test;
Friedreich's Ataxia Rating Scale-Activities of Daily Living (FARS_ADL);

To evaluate the effect of long-term SC administration of CTI-1601 on other clinical evaluations of FRDA as assessed by:
Modified Fatigue Impact Scale (MFIS);
Functional Staging for Ataxia;
Patient Global Impression of Change (PGI-C);
Clinical Global Impression of Change (CGI-C);
Left ventricular ejection fraction (LVEF);
LV end-diastolic volume (EDV);
LV mass;
LV mass index (LVMi); and
Hemoglobin Alc (HbAlc).

To evaluate the safety and tolerability of long-term SC administration of CTI-1601 in subjects with FRDA.

To evaluate the PK of long-term SC administration of CTI-1601.

### Exploratory Objectives

To characterize the immunogenicity of long-term SC administration of CTI-1601; and
To evaluate the effect of long-term SC administration of CTI-1601 on gene expression and selected lipids.

### Study Endpoints

### Primary Endpoints

Change from baseline at each collection timepoint in tissue FXN concentrations normalized to total protein (tissue FXN ratio) observed in:
Buccal cells collected from cheek swabs; and
Skin cells collected from skin punch biopsies.

### Secondary Endpoints

Change from baseline in the mFARS and upright stability subscale examinations;
Change from baseline in the 9-HPT;
Change from baseline in the T25-FW test;
Change from baseline in the FARS-ADL;
Change from baseline in the MFIS;
Change from baseline in Functional Staging for Ataxia;
Overall impression of change as assessed by the subject using the PGI-C;
Overall impression of change as assessed by the clinician using the CGI-C;
Observed and change from baseline in LVEF, LV EDV, LV mass, and LVMi obtained from echocardiogram (ECHO);
Observed and change from baseline in HbAlc laboratory results;
Number of AEs, TEAEs, study drug-related TEAEs, Grade 3/4 TEAEs, and SAEs;
Change from baseline in clinical laboratory tests;
Observed and change from baseline in electrocardiogram (ECG) parameters;
Observed and change from baseline in ECHO parameters;
Observed and change from baseline in vital signs (VS);
Number of subjects with abnormal physical examination parameters;
Number of subjects who experience Suicidal Ideation and/or Suicidal Behavior based on the C-SSRS;
PK parameters during a dosing interval including, but not limited to, the following:
   AUC₀₋ₜ: Area under the concentration-time curve from time 0 to the time of last quantifiable concentration;
   Cₘₐₓ: Maximum observed concentration;
   Tₘₐₓ: Time of maximum observed concentration; and
   C_{trough}: Concentration reached immediately before the next dose is administered.

### Exploratory Endpoints

Change from baseline in gene expression in buccal cells collected from cheek swabs;
Change from baseline in lipids in plasma derived from whole blood; and
Incidence of anti-drug antibodies (ADAs) to CTI-1601.

### Study Design

All subjects will receive CTI-1601 in this study. To provide for a surrogate placebo comparator group, an external control group will be constructed from data collected from participants in the Friedreich's Ataxia Clinical Outcome Measures Study (FACOMS), a natural history study for FRDA that collects information from an annual evaluation that includes the mFARS and upright stability subscale examinations, the T25-FW test, FARS-ADL, and the 9-HPT, if enough data are available from the natural history study.

Subjects who complete a prior study of CTI-1601, regardless of whether the subject took CTI-1601 or placebo, will be eligible to participate in this study unless the subject experienced one or more of the following in a previous CTI-1601 study:
SAE related to study drug;
Significant AE, defined as Grade 3 or higher according to the CTCAE, Version 5.0 or higher, related to study drug;
Some other event, related to participation in a previous study with CTI-1601, that supports the exclusion of the subject from participating in this study as determined by the sponsor (i.e., an AE considered clinically significant by the sponsor regardless of whether it met SAE criteria and regardless of CTCAE grade);
Withdrawal from participation in a previous study of CTI-1601 for any reason

A subject will not be screened until all assessments pertaining to the prior study have been completed.

Concomitant use of omaveloxolone (an approved treatment for patients with FRDA) will be permitted in this study. Subjects who are currently receiving omaveloxolone or intend to receive omaveloxolone must either receive CTI-1601 for 6 months prior to their first dose of omaveloxolone or receive omaveloxolone for 6 months prior to their first dose of CTI-1601. Subjects already receiving omaveloxolone must provide documentation of their first pharmacy fulfillment date for omaveloxolone. This date will be used as the start date.
If the omaveloxolone fulfillment date is greater than or equal to 6 months relative to their estimated Day 1 visit for this study, the subjects will proceed with the screening assessments described beginning with Day-42.
If the omaveloxolone fulfillment date is less than 6 months relative to their estimated Day 1 visit for this study, the subject will participate in a screening period of up to 6 months. This will include monthly calls from study staff to collect information on Aes and ongoing medications until 2 months prior to initiating treatment with CTI 1601. Subjects will then proceed with the Screening visit described for Day-30.

Subjects who are not receiving omaveloxolone at the Screening visit for this study will not be permitted to use omaveloxolone concomitantly until after they have completed 6 months of treatment with CTI-1601. Subjects will proceed with the screening assessments described beginning with Day-42.

Screening assessments will be performed by a combination of on-site and telehealth (TH) visits. On-site Screening assessments (safety laboratory tests, physical examination, ECG, and ECHO) must be completed with results available within 30 days of Day 1. In addition, a TH visit will be completed within approximately 2 weeks of the Day-1 visit. A freezer and study drug will be sent to the subject prior to the subject traveling to the site for the Day-1 visit. At the Day 1 visit, the study site or a trained subject/caregiver will prepare and administer the first dose of CTI-1601 to eligible subjects. Subjects will receive training to self-administer CTI-1601 SC if the subject has the dexterity and visual acuity to perform SC injections; otherwise, a caregiver committed to preparing and administering the injections will be trained. A back-up caregiver(s) may also be trained. Anatomic areas used as injection sites will be assessed at each scheduled visit and any associated AEs will be recorded as appropriate.

Subject safety will be assessed monthly via remote contact (TH) or on-site clinic visits for the duration of the study. Most of the efficacy assessments will be completed every 3 months (Q3M) starting at Day 90. Two Follow-up visits (after a subject discontinues study drug entirely [see below]) will be performed by a visiting nurse 7 days and 30 days post last dose with a window of +3 days. All visits will occur at the study site except for the TH visits (which will occur between on-site visits), and the Follow-up visits will be completed by the visiting nurse and by study site staff via TH.

At the initiation of the OLE study described in Example 4, CTI-1601 25 mg was administered SC QD each morning starting on Day 1. Under this amended protocol, CTI-1601 50 mg will be administered SC QD each morning based on the subject's status below:
New subjects enrolled and subjects currently in screening who have not had their Day 1 visits will initiate dosing with CTI-1601 50 mg administered SC QD each morning.
Subjects receiving CTI-1601 25 mg for <60 days will initiate dosing with CTI-1601 50 mg during the Day 60 on-site visit.
Subjects receiving CTI-1601 25 mg for >60 days but <90 days will initiate dosing with CTI-1601 50 mg during the Day 90 on-site visit.
Subjects receiving CTI-1601 25 mg for >90 days will initiate dosing with CTI-1601 50 mg either at the next scheduled on-site visit or at an additional on-site visit approximately 30 days prior to the next scheduled on-site visit.

For subjects transitioning from CTI-1601 25 mg to CTI-1601 50 mg, safety laboratory evaluations (blood samples for hematology and chemistry) will be performed approximately 14 days after initiating CTI-1601 50 mg.

Study drug (CTI-1601) will be administered SC QD. The concentration of the CTI-1601 dose formulation for SC injection is 50 mg/mL. The dose planned for this study appears to have been safe and well tolerated in patients with FRDA who participated in previous clinical studies.

### Study Drug

CTI-1601 is a recombinant fusion protein consisting of a cell penetrant peptide linked to the complete FXN protein. CTI-1601 will be provided in liquid form in a 2.0 mL vial with each vial containing 1.25 mL of product at a concentration of 50 mg/mL. CTI-1601 contains the following excipients: histidine, sucrose, polysorbate 20, and sterile water.

CTI-1601 will be administered SC QD, via syringe filled by trained subject/caregiver to contain the appropriate amount to be administered.

Study drug (50 mg CTI-1601) administration will occur QD at approximately the same time in the morning on each dosing day.

### Study Evaluations and Procedures

See section entitled “Study Evaluations and Procedures” in Example 4.

### Informal Sequence Listing

(full-length human FXN protein)
SEQ ID NO: 1
MWTLGRRAVAGLLASPSPAQAQTLTRVPRPAELAPLCGRRGLRTDIDATC
TPRRASSNQRGLNQIWNVKKQSVYLMNLRKSGTLGHPGSLDETTYERLAE
ETLDSLAEFFEDLADKPYTFEDYDVSFGSGVLTVKLGGDLGTYVINKQTP
NKQIWLSSPSSGPKRYDWTGKNWVYSHDGVSLHELLAAELTKALKTKLDL
SSLAYSGKDA
(mature human FXN protein)
SEQ ID NO: 2
SGTLGHPGSLDETTYERLAEETLDSLAEFFEDLADKPYTFEDYDVSFGSG
VLTVKLGGDLGTYVINKQTPNKQIWLSSPSSGPKRYDWTGKNWVYSHDGV
SLHELLAAELTKALKTKLDLSSLAYSGKDA
(mitochondrial targeting sequence of human FXN
protein)
SEQ ID NO: 3
MWTLGRRAVAGLLASPSPAQAQTLTRVPRPAELAPLCGRRGLRTDIDATC
TPRRASSNQRGLNQIWNVKKQSVYLMNLRK
(HIV-TAT peptide)
SEQ ID NO: 4
YGRKKRRQRRR
(TAT-FXN fusion protein)
SEQ ID NO: 5
MYGRKKRRQRRRGGMWTLGRRAVAGLLASPSPAQAQTLTRVPRPAELAPL
CGRRGLRTDIDATCTPRRASSNQRGLNQIWNVKKQSVYLMNLRKSGTLGH
PGSLDETTYERLAEETLDSLAEFFEDLADKPYTFEDYDVSFGSGVLTVKL
GGDLGTYVINKQTPNKQIWLSSPSSGPKRYDWTGKNWVYSHDGVSLHELL
AAELTKALKTKLDLSSLAYSGKDA
(peptide derived from mature human FXN protein)
SEQ ID NO: 6
SGTLGHPGSLDETTYER
(peptide derived from mature human FXN protein)
SEQ ID NO: 7
LGGDLGTYVINK

1. A method of increasing level of frataxin (FXN) protein in an FXN deficient subject, said method comprising administering to said subject a TAT-FXN fusion protein at a dose of about 25 mg;
wherein said dose is administered once daily for at least 14 days;
and wherein said TAT-FXN fusion protein comprises the amino acid sequence set forth in SEQ ID NO: 5;
such that the level of FXN protein in a solid tissue sample obtained from said subject following administration of the TAT-FXN fusion protein is increased as compared to the level of FXN protein prior to the administration of the TAT-FXN fusion protein.

2. The method of claim 1, wherein said solid tissue sample is a skin biopsy or a buccal tissue.

3. (canceled)

4. The method of claim 1, wherein said TAT-FXN fusion protein is administered to said subject for at least 30 days, at least 60 days, or at least 90 days.

5.-6. (canceled)

7. The method of claim 1, wherein the level of FXN protein in a solid tissue sample obtained from said subject following administration of the TAT-FXN fusion protein is increased by at least about 10%, at least about 20%, at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%.

8. The method of claim 1, wherein the level of FXN protein in a solid tissue sample obtained from said subject following administration of the TAT-FXN fusion protein is increased to at least about 12.5%, at least about 25%, at least about 37.5%, at least about 50% or at least about 75% of the level of FXN protein in a healthy subject.

9. The method of claim 1,
wherein the level of FXN protein in a buccal tissue sample following administration of the TAT-FXN fusion protein for at least 30 days is about 0.5 to about 3.0 pg/μg of total protein;
wherein the level of FXN protein in a buccal tissue sample following administration of the TAT-FXN fusion protein for at least 60 days is about 1.0 to about 3.3 pg/μg of total protein; or
wherein the level of FXN protein in a buccal sample following administration of the TAT-FXN fusion protein for at least 90 days is about 1.4 to about 3.7 pg/μg of total protein.

10.-11. (canceled)

12. The method of claim 1, wherein the level of FXN protein in a buccal tissue sample following administration of the TAT-FXN fusion protein for at least 30 days is increased by about 0.01 to about 1.5 pg/μg of total protein, as compared to the level of FXN protein prior to administration of the TAT-FXN fusion protein;
wherein the level of FXN protein in a buccal tissue sample following administration of the TAT-FXN fusion protein for at least 60 days is increased by about 0.10 to about 1.8 pg/μg of total protein, as compared to the level of FXN protein prior to administration of the TAT-FXN fusion protein; or
wherein the level of FXN protein in a buccal tissue sample following administration of the TAT-FXN fusion protein for at least 90 days is increased by about 0.5 to about 2.6 pg/μg of total protein, as compared to the level of FXN protein prior to administration of the TAT-FXN fusion protein.

13.-14. (canceled)

15. The method of claim 1, wherein the level of FXN protein in a buccal tissue sample following administration of the TAT-FXN fusion protein is increased to at least about 30% of the level of FXN protein in a healthy subject following administration of the TAT-FXN fusion protein for at least 30 days, at least 60 days, or at least 90 days.

16. The method of claim 1, wherein the level of FXN protein in a skin biopsy sample following administration of the TAT-FXN fusion protein for at least 30 days ranges from about 4.5 to about 8.5 pg/μg of total protein; or
wherein the level of FXN protein in a skin biopsy sample following administration of the TAT-FXN fusion protein for at least 90 days ranges from about 7.0 to about 10.5 pg/μg of total protein.

17. (canceled)

18. The method of claim 1, wherein the level of FXN protein in a skin biopsy sample following administration of the TAT-FXN fusion protein for at least 30 days is increased by about 1.5 to about 6.0 pg/μg of total protein, as compared to the level of FXN protein prior to administration of the TAT-FXN fusion protein; or
wherein the level of FXN protein in a skin biopsy sample following administration of the TAT-FXN fusion protein for at least 90 days is increased by about 4.2 to about 9.0 pg/μg of total protein, as compared to the level of FXN protein prior to administration of the TAT-FXN fusion protein.

19. (canceled)

20. The method of claim 1, wherein the level of FXN protein in a skin biopsy sample following administration of the TAT-FXN fusion protein is increased to at least about 45% of the level of FXN protein in a healthy subject following administration of the TAT-FXN fusion protein for at least 30 days, or increased to at least about 70% of the level of FXN protein in a healthy subject following administration of the TAT-FXN fusion protein for at least 90 days.

21. A method of treating Friedreich's Ataxia in a subject in need thereof, said method comprising administering to said subject a TAT-FXN fusion protein at a dose of about 25 mg;
wherein said dose is administered once daily for at least 14 days;
and wherein said TAT-FXN fusion protein comprises the amino acid sequence set forth in SEQ ID NO: 5;
such that the level of FXN protein in a solid tissue sample obtained from said subject following administration of the TAT-FXN fusion protein is increased as compared to the level of FXN protein prior to the administration of the TAT-FXN fusion protein.

22. The method of claim 21, wherein said solid tissue sample is a skin biopsy or a buccal tissue.

23. (canceled)

24. The method of claim 21, wherein said TAT-FXN fusion protein is administered to said subject for at least 30 days, at least 60 days, or at least 90 days.

25.-40. (canceled)

41. A method of increasing level of frataxin (FXN) protein in an FXN deficient subject, said method comprising administering to said subject a TAT-FXN fusion protein at a dose of about 50 mg;
wherein said dose is administered once daily for at least 14 days;
and wherein said TAT-FXN fusion protein comprises the amino acid sequence set forth in SEQ ID NO: 5;
such that the level of FXN protein in a solid tissue sample obtained from said subject following administration of the TAT-FXN fusion protein is increased by at least about 20% as compared to the level of FXN protein prior to the administration of the TAT-FXN fusion protein.

42. The method of claim 41, wherein said solid tissue sample is a skin biopsy or a buccal tissue.

43. (canceled)

44. The method of claim 41, wherein said TAT-FXN fusion protein is administered to said subject for at least 3 weeks, at least 4 weeks, at least 5 weeks, at least 6 weeks, at least 7 weeks, at least 8 weeks, at least 9 weeks, at least 10 weeks, or at least 12 weeks.

45. (canceled)

46. The method of claim 41, wherein the level of FXN protein in a solid tissue sample obtained from said subject following administration of the TAT-FXN fusion protein is increased by at least about 30%, at least about 40%, at least about 50%, at least about 60%, at least about 70%, at least about 80%, at least about 90%, at least about 100%, at least about 250%, at least about 500%, at least about 600%, at least about 700%, at least about 800%, at least about 900%, at least about 1000%, at least about 1500%, at least about 2000%, at least about 2500%, at least about 5000% or at least about 10000%.

47. The method of claim 41, wherein the level of FXN protein in a solid tissue sample obtained from said subject following administration of the TAT-FXN fusion protein is increased to at least about 25%, at least about 37.5%, at least about 50%, or at least about 70% of the level of FXN protein in a healthy subject.

48. A method of treating Friedreich's Ataxia in a subject in need thereof, said method comprising administering to said subject a TAT-FXN fusion protein at a dose of about 50 mg;
wherein said dose is administered once daily for at least 14 days;
and wherein said TAT-FXN fusion protein comprises the amino acid sequence set forth in SEQ ID NO: 5;
such that the level of FXN protein in a solid tissue sample obtained from said subject following administration of the TAT-FXN fusion protein is increased by at least about 20% as compared to the level of FXN protein prior to the administration of the TAT-FXN fusion protein.

49. The method of claim 48, wherein said solid tissue sample is a skin biopsy or a buccal tissue.

50-54. (canceled)
